(19) United States
(12) Patent Application Publication
(10) Pub. No.: US 2024/0157042 A1
(43) Pub. Date: May 16, 2024

(54) POLE SUPPORT SYSTEMS AND METHODS

(71) Applicants: MARK SUNDERLAND, OTTAWA (CA); GAIL SNUGGS, OTTAWA (CA)

(72) Inventors: MARK SUNDERLAND, OTTAWA (CA); GAIL SNUGGS, OTTAWA (CA)

(21) Appl. No.: 18/548,589

(22) PCT Filed: Mar. 3, 2022
(86) PCT No.: PCT/CA2022/050300

§ 371 Date: Sep. 1, 2023

Related U.S. Application Data
(60) Provisional application No. 63/156,596, filed on Mar. 4, 2021.

(51) Int. Cl. A61M 5/14

(52) U.S. Cl. CPC A61M 5/1417; A61M 5/1415

(57) ABSTRACT

A patient may be connected to a variety of medical equipment which may vary patient to patient or during the patient's treatment. Similarly, other user may wish to have configurable workstations, configurable desks, configurable displays, configurable furniture etc. where in each instance, both medical and non-medical, the system should not only provide mounting and support but also ease of mobility. The invention provides a pole support system with attended attachments allowing a diverse range of equipment, supports, shelves, electrical equipment etc. to be mounted and supported upon a mobile base in a highly configurable manner. Further, a user can easily move and position some elements upon the pole support system with only one hand as their other hand is engaged holding something else.

### CROSS-REFERENCE TO RELATED APPLICATIONS

This patent application claims the benefit of priority as a 371 National Phase Entry application of PCT/CA2022/050300 filed 3 Mar. 2022; which itself claims the benefit of priority from U.S. Provisional Patent Application 63/156,596 filed 4 Mar. 2021; the entire contents of each being incorporated herein by reference.

### FIELD OF THE INVENTION

This patent application relates to pole support systems and more particularly to brackets, supports, poles, and bases for pole support systems.

### BACKGROUND OF THE INVENTION

Today a patient whether on a ward pre-surgery, in an operating theater during surgery, on a ward post-surgery, in an emergency room, on a ward for recover or observation may be connected to a variety of medical equipment such as intravenous (IV) drip(s), infusion pump(s), heart monitor, continuous positive airway pressure (CPAP) machine, blood pumps, etc. This equipment must be mounted and supported plus in most instances be mobile such that it can be either moved into place, moved away, or moved with the patient.

Accordingly, it would be beneficial to provide hospital, clinics, and other medical environments with a pole support system allowing this diverse range of medical equipment to be mounted/supported upon a mobile base in a highly configurable manner. It would be further beneficial for the pole support system to enable medical personnel to easily move and position items upon the pole support system, ideally with only one hand as their other hand is generally engaged.

Within other applications it would be beneficial to provide users with a pole support system allowing shelves and other elements to be mounted/supported upon a mobile base in a highly configurable manner. It would be further beneficial for the pole support system to enable a user to easily move and position items upon the pole support system, ideally with only one hand as their other hand is generally engaged.

Other aspects and features of the present invention will become apparent to those ordinarily skilled in the art upon review of the following description of specific embodiments of the invention in conjunction with the accompanying figures.

### SUMMARY OF THE INVENTION

It is an object of the present invention to mitigate limitations within the prior art relating to pole support systems and more particularly to brackets, supports, poles, and bases for pole support systems.

In accordance with an embodiment of the invention there is provided a non-removable knob assembly comprising:
a fixing which terminates with a threaded portion at one end;
a knob comprising a body with a first opening through the knob such that the knob fits over the threaded portion of the fixing, a second opening disposed at a first end of the knob nearest the terminal end of the threaded portion of the fixing, and a threaded portion disposed at a second distal end to the first end;
a locknut attached to the terminal end of the threaded portion; wherein
rotation of the knob in a first direction results in the knob moving along the threaded portion of the fixing away from the terminal end of the threaded portion of the fixing;
rotation of the knob in a second direction results in the knob moving along the threaded portion of the fixing towards the terminal end of the threaded portion of the fixing; and the locknut limits movement of the knob when rotated in the second direction.

In accordance with an embodiment of the invention there is provided a mount comprising:
a body with a threaded opening through which a threaded element passes;
a knob attached to a first end of the threaded element;
a strap disposed within the body attached to a second distal end of the threaded element and surrounding a first pole passing through the mount;
the strap is tightened to retain the mount in position through rotation of the knob moving the threaded element relative to the body;
the strap is loosened through rotation of the knob moving the threaded element relative to the body thereby allowing the mount to be moved upon the first pole.

In accordance with an embodiment of the invention there is provided a mount comprising:
a body with a threaded opening through which a threaded element passes, a pair of openings for a pole to fit through the body and another pair of openings for another pole to fit through the body;
a knob attached to a first end of the threaded element;
an engagement plate attached at a second distal end of the threaded element;
a retainer attached to the body at one end and having a predetermined geometry at a second distal end;
a spring disposed between a block and an auxiliary block; wherein
operation of the knob moves the engagement plate relative to the retainer to either hold or release a first pole inserted through the pair of openings and disposed between the engagement plate and an inner surface of the retainer; and
the block, spring and auxiliary block retain the another pole inserted through the other pair of openings independent of the position of the knob.

In accordance with an embodiment of the invention there is provided a support comprising:
providing a shell having an opening of predetermined diameter disposed at a first end to fit over providing a pole;
providing a fitting disposed at a second distal end to provide a specific function; and
providing a split ring disposed within a groove around the opening within the shell; wherein
the split ring projects into the opening and has an inner opening having a diameter established in dependence upon the diameter of the pole; and
the support is fitted onto the pole by inserting the pole through the opening and is retained in position on the pole independent of any other means or element.

In accordance with an embodiment of the invention there is provided a base comprising:
a first arm for attaching to a bar in a first predetermined location;
a second arm for attaching to the bar in a second predetermined location;
the bar having one or more mounting points;
a first pair of wheels attached to third predetermined locations relative to each end of the first arm;
a second pair of wheels attached to fourth predetermined locations relative to each end of the second arm; and
a third pair of wheels attached to fifth predetermined locations relative to each end of the arm;
wherein
the third predetermined locations, the fourth predetermined locations, and fifth predetermined locations are all points on a common circle; and
each of the one or more mounting points is for mounting a pole.

In accordance with an embodiment of the invention there is provided a method of providing a pole support system comprising:
providing a base;
providing at least one pole of a plurality of poles attached to the base; and
providing at least one support of a plurality of supports mounted to the plurality of poles.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 depicts an exemplary deployment scenario for pole support systems (POLSUS) according to embodiments of the invention;

FIG. 2A depicts an exemplary single pole POLSUS configuration according to an embodiment of the invention;

FIG. 2B depicts an exemplary double pole POLSUS configuration according to an embodiment of the invention;

FIG. 2C depicts an exemplary triple pole POLSUS configuration according to an embodiment of the invention;

FIGS. 3A and 3B depict plan and bottom views of an exemplary base configuration for POLSUS configurations according to embodiments of the invention;

FIG. 4 depicts an exemplary POLSUS configuration according to embodiments of the invention;

FIGS. 5 and 6 depict exemplary demonstrations of compact high density storage of POLSUS configurations according to embodiments of the invention exploiting a base such as depicted in FIGS. 3A and 3B respectively;

FIG. 7 depicts a perspective view of an exemplary modular POLSUS system providing a single pole POLSUS configuration according to an embodiment of the invention;

FIG. 8 depicts a perspective view of an exemplary modular POLSUS system providing a double pole POLSUS configuration according to an embodiment of the invention;

FIG. 9 depicts a perspective view of an exemplary modular POLSUS system providing a triple pole POLSUS configuration according to an embodiment of the invention;

FIGS. 10A to 10C depict cross-sectional elevation views of exemplary modular POLSUS systems providing configurable POLSUS systems according to embodiments of the invention;

FIG. 11 depicts partial exploded schematics of a pole assembly mechanism for modular POLSUS systems providing configurable POLSUS systems according to embodiments of the invention;

FIGS. 12A and 12B depict exemplary pole supports providing a tube retainer and hook according to embodiments of the invention for attachment to a POLSUS configuration according to embodiments of the invention;

FIGS. 13A and 13B depict a detail and assembly image for an exemplary pole support according to an embodiment of the invention;

FIGS. 14A and 14B depict a detail and assembly image for an exemplary pole support according to an embodiment of the invention;

FIG. 14C depicts an assembly image for another exemplary pole support according to an embodiment of the invention;

FIG. 15 depicts details of a body portion of an exemplary pole support according to the embodiments of the invention depicted in FIGS. 7 to 9 respectively;

FIGS. 16 and 17 depict the exemplary pole support according to the embodiments of the invention depicted in FIGS. 12A to 15 respectively in movable and locked configurations respectively;

FIG. 18 depicts additional equipment for use in conjunction with POLSUS according to embodiments of the invention;

FIG. 19 depicts exemplary configurations for the pole support arms for assembly with a pole support body according to embodiments of the invention;

FIGS. 20A and 20B depict an adjustable resistance knob with retaining mechanism according to an embodiment of the invention;

FIG. 21 depicts a connection hub for use with pole systems according to embodiments of the invention, in this instance a handle grab;

FIG. 22 depicts the connection hub of FIG. 21 for use with pole systems according to embodiments of the invention, in this instance for an adjustable platform;

FIGS. 23A and 23B depict a side arm to support an auxiliary pole for use with pole systems according to embodiments of the invention;

FIG. 24 depicts another side arm to support an auxiliary pole for use with pole systems according to embodiments of the invention;

FIG. 25A depicts non-deployed and deployed configurations for an anti-nesting element for use with pole systems according to embodiments of the invention;

FIG. 25B depicts an exemplary design of an anti-nesting element according to an embodiment of the invention;

FIGS. 26A and 26B depict exemplary designs of an anti-nesting element according to an embodiment of the invention; and

FIG. 27 depicts an exemplary power entry/exit with cover for distributing power up a POLSUS according to an embodiment of the invention.

### DETAILED DESCRIPTION

The present invention is direct to pole support systems and more particularly to brackets, supports, poles, and bases for pole support systems.

The ensuing description provides representative embodiment(s) only, and is not intended to limit the scope, applicability or configuration of the disclosure. Rather, the ensuing description of the embodiment(s) will provide those skilled in the art with an enabling description for implementing an embodiment or embodiments of the invention. It being understood that various changes can be made in the function and arrangement of elements without departing from the spirit and scope as set forth in the appended claims. Accordingly, an embodiment is an example or implementation of the inventions and not the sole implementation. Various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention can also be implemented in a single embodiment or any combination of embodiments.

Reference in the specification to “one embodiment,” “an embodiment,” “some embodiments” or “other embodiments” means that a particular feature, structure, or characteristic described in connection with the embodiments is included in at least one embodiment, but not necessarily all embodiments, of the inventions. The phraseology and terminology employed herein is not to be construed as limiting but is for descriptive purpose only. It is to be understood that where the claims or specification refer to “a” or “an” element, such reference is not to be construed as there being only one of that element. It is to be understood that where the specification states that a component feature, structure, or characteristic “may,” “might,” “can” or “could” be included, that particular component, feature, structure, or characteristic is not required to be included.

Reference to terms such as “left,” “right,” “top,” “bottom,” “front” and “back” are intended for use in respect to the orientation of the particular feature, structure, or element within the figures depicting embodiments of the invention. It would be evident that such directional terminology with respect to the actual use of a device has no specific meaning as the device can be employed in a multiplicity of orientations by the user or users.

Reference to terms “including,” “comprising,” “consisting” and grammatical variants thereof do not preclude the addition of one or more components, features, steps, integers or groups thereof and that the terms are not to be construed as specifying components, features, steps or integers. Likewise, the phrase “consisting essentially of,” and grammatical variants thereof, when used herein is not to be construed as excluding additional components, steps, features integers or groups thereof but rather that the additional features, integers, steps, components or groups thereof do not materially alter the basic and novel characteristics of the claimed composition, device or method. If the specification or claims refer to “an additional” element, that does not preclude there being more than one of the additional element.

A “pole support system” (POLSUS) as used herein and throughout this disclosure, refers to a system comprising one or more upright members, e.g. poles, attached to a mobile base or fixed to an item of infrastructure in conjunction with one or more pole supports which are fitted over the upright member(s) allowing them to be moved relative to the upright member(s) and providing a specific function.

A “fitting” as used herein and throughout this disclosure, refers to any machine component, piping or tubing part that can attach or connect two or more parts. Such fittings may include, but not be limited to, a coupling, couplings, compression fitting, pipe fitting, piping fittings, plumbing fittings, and plumbing fitting.

A “mounting” as used herein and throughout this disclosure, refers to part of a device, system, ancillary, etc. which is configured to support and/or attach another device, system, ancillary, components etc. to said part of the device, system, ancillary, component etc. A mounting typically supports demountable attachment of the parts but may be employed in permanent attachment to define the location of the point of attachment or support demountable attachment prior to permanent attachment.

A “fixing” or “attachment means” as used herein and throughout this disclosure, refers to component, device, or means employed to permanently or demountably attach a device, system, ancillary, components etc. to part of another device, system, ancillary, component etc. This may include, but not be limited to, depending upon whether permanent or demountable and the material(s) being joined fasteners, glues, resins, epoxies, cementing, welding, soldering, brazing, pressure differentials, magnets, clamps, clips, ties, supports, physical retention elements such as clips and crimps, and physical retention methods such as friction and interference fit. Fasteners may include, but not be limited to, bolts, nuts, washers, screws, threaded fasteners, rivets, nails, pins, hook-and-eye, and hook and loop.

A “demountable” connection as used herein and throughout this disclosure, refers to component, device, or means employed to permanently or demountably attach an electrical connection or fluidic connection on a device, system, ancillary, components etc. to another electrical connection or fluidic connection on another device, system, ancillary, component etc. Electrical “demountable” connections are typically formed by plug and socket arrangements in discrete, linear array, or two-dimensional (2D) array formats or discrete male-female threaded connectors typically employed for microwave and RF. Fluidic “demountable” connections typically are formed by male-female threaded connectors with O-ring, sealing ring or gasket seals.

A “fluid” as used herein refers to a liquid, a gas, a mixture of liquids or a mixture of gases.

A “portable electronic device” (PED) as used herein and throughout this disclosure, refers to a wireless device used for communications and other applications that requires a battery or other independent form of energy for power. This includes devices, but is not limited to, such as a cellular telephone, smartphone, personal digital assistant (PDA), portable computer, pager, portable multimedia player, portable gaming console, laptop computer, tablet computer, a wearable device and an electronic reader.

A “fixed electronic device” (FED) as used herein and throughout this disclosure, refers to a wireless and/or wired device used for communications and other applications that requires connection to a fixed interface to obtain power. This includes, but is not limited to, a laptop computer, a personal computer, a computer server, a kiosk, a gaming console, a digital set-top box, an analog set-top box, an Internet enabled appliance, an Internet enabled television, and a multimedia player.

Within the following description embodiments of the invention are described and depicted with respect to medical pole supports. However, it would be evident to one of skill in the art that the embodiments of the invention may be employed within other environments and applications including, for example, configurable workstations, configurable desks, configurable displays, configurable furniture.

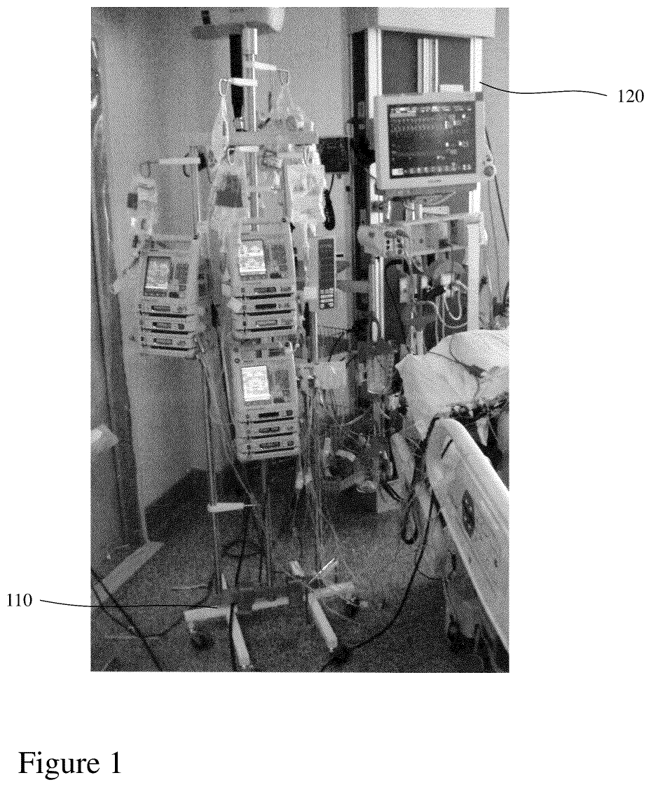

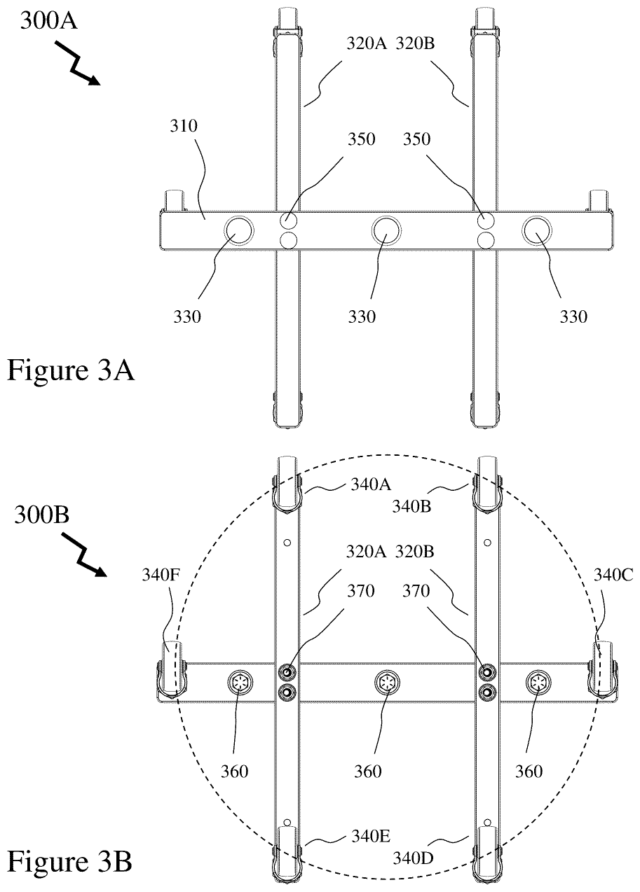

Referring to FIG. 1 depicts an exemplary deployment scenario for pole support systems (POLSUS) according to embodiments of the invention. Accordingly, multiple items of medical equipment are attached to a mobile Pole Support System (POLSUS) 110 and a fixed POLSUS 120. POLSUS 110 being a triple pole POLSUS such as depicted in FIG. 4. Attached to the POLSUS 110 are a series of pole supports (not identified for clarity) which provide for electrical cable management, fluid tube management, IV drip mounting, infusion pump mounting, and a control interface. As depicted POLSUS 110 is configured with 3 infusion pumps and 7 IV drip bags. Accordingly, it would be evident that a POLSUS should support both very simple configurations (e.g. a single IV drip that a patient can move with themselves on a single pole) and very complex configurations (e.g. post-surgery or during surgery with multiple poles and multiple pumps).

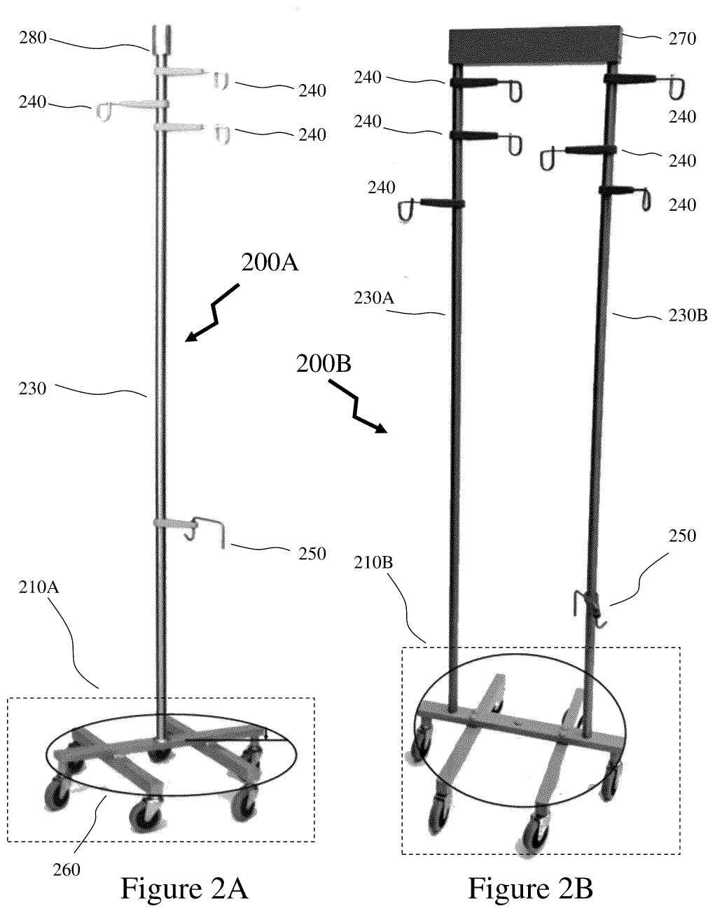

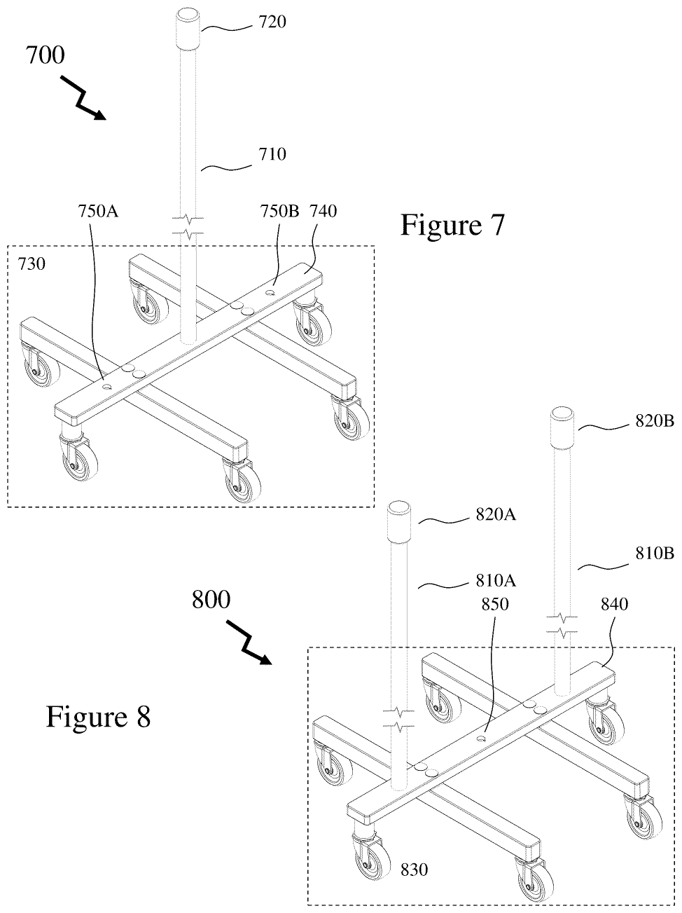

FIGS. 2A to 2C depict exemplary POLSUS 200A to 200C configurations exploiting pole supports and POLSUS bases according to embodiments of the invention. Referring initially to FIG. 2A there is depicted a single pole POLSUS 200A comprising a wheeled POLSUS base 210A with a single Pole 230. Attached to the Pole 230 are 3 Fluid Support Holders (FLSH) pole supports 240 disposed towards the top and a single hook pole support 250. The hook pole support 250 allows for its end distal to the Pole 230 to be “hooked” onto a wheelchair, for example, such that the first POLSUS 200A moves automatically, for example in conjunction with a wheelchair or bed, as opposed to being pulled or “hooked” to a bed/chair/wall etc. such that the first POLSUS 200A cannot be accidentally moved. As will become evident in respect of the description below in respect of FIGS. 7 to 11 the pole supports such as hook pole support 250 and FLSH pole supports 240 can be moved relative to the POLSUS 200A by an individual with a single hand providing increased usability, flexibility etc. It would be evident that the number of FLSH pole supports 240 may vary from 1 to a relatively significant number such as 4, 5, 6, 9, 12, 15 or more for example.

Referring to FIG. 2B with second POLSUS 200B this similarly comprises the wheeled POLSUS base 210 with a first and second single poles 230A and 230B respectively. As depicted the second POLSUS 200B comprises a wheeled POLSUS base 210A to which are attached first and second poles 230A and 230B respectively. Attached to the first Pole 230A are three FLSH pole supports 240 whilst the second Pole 230B has a hook pole support 250 and a further 3 FLSH pole supports 240. The wheeled POLSUS base 230B allowing the second POLSUS 200B to be moved with ease by a patient or other individual whilst the hook pole support 250 allows it to be attached to fixed or mobile elements. Attached to the top of the first and second poles 230A and 230B respectively is a joining element 270 which may be employed to increase the rigidity of each of the first and second poles 230A and 230B respectively by linking them and/or provide for identification of the POLSUS 200B, e.g. infirmary, neo-natal, surgical, etc. or allow for the provisioning of advertising or other information at the top of the POLSUS 200B. Accordingly, the Joining Element 270 may comprise a display and ancillary electronics, wireless interface, etc. This information may be programmable, periodically updated, or continuously updated. Optionally, the information may be obtained from sensors or controllers associated with POLSUS such as those providing temperature, relative humidity, a status of equipment attached to the POLSUS, a fill state of an IV bag attached to the POLSUS, etc. It would be evident that the number of FLSH pole supports 240 may vary from 1 to a relatively significant number such as 4, 5, 6, 9, 12, 15 or more for example.

Now referring to third POLSUS 200C in FIG. 2C there are depicted first to third poles 230A to 230C respectively which are attached to a wheeled MESDSUS base 210C. The first Pole 230A has 3 FLSH pole supports 240 whilst second Pole 230B has a further three FLSH pole supports 240 and a hook pole support 250. The third pole having a further 3 FLSH pole supports 240. As with POLSUS 200B the first to third poles 230A to 230C respectively are coupled together via joining element 270 at their top. Optionally, as noted above the Joining Element 270 allows for identification information to be displayed such as, for example within a hospital, a sponsor, a ward identity, a surgery room identity, etc. or to display advertising etc.

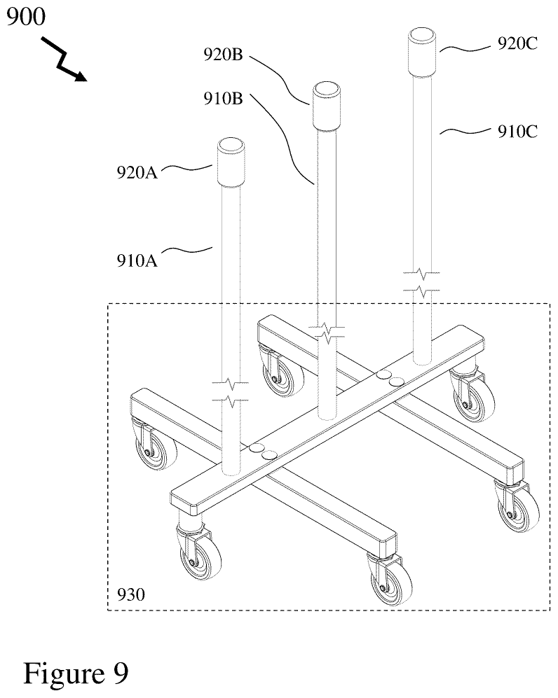

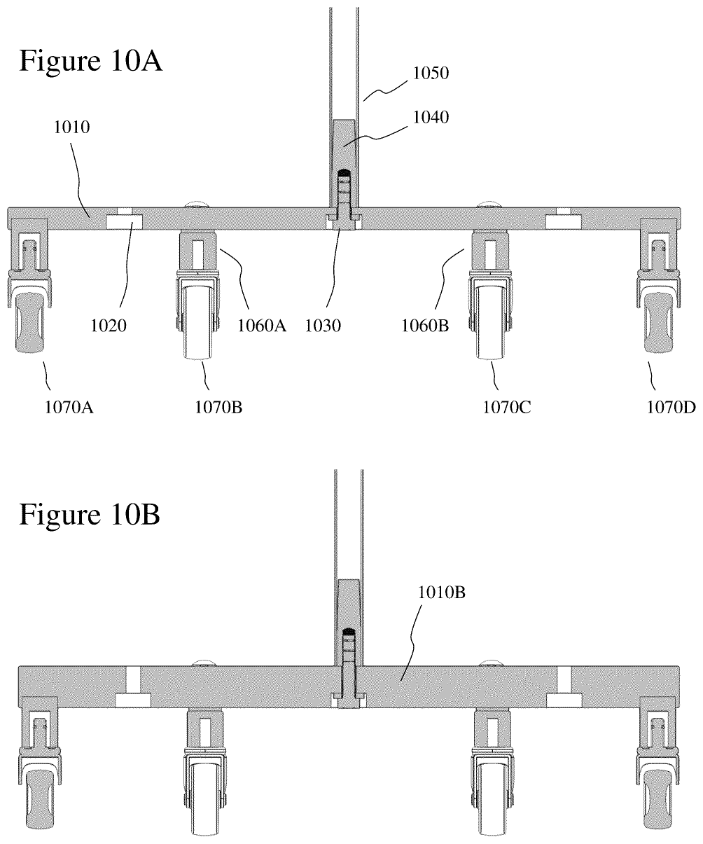

With the second and third POLSUS 200B and 200C the Joining Element 270 may be attached in a demountable manner allowing the pole supports mounted to the different poles to be reconfigured or changed. A top element 280 is employed with first POLSUS 200A such that the pole supports are retained onto the pole unless the top element 280 is removed. However, the top element 280 also provides a cap for the pole such that if tubing is employed the hollow inner of the pole does not accumulate dust, debris etc. The same benefit arises from the Joining Element 270 on the second and third POLSUS 200B and 200C respectively. However, as depicted in FIGS. 8 and 9 discrete top elements may be employed on each pole of the dual and triple pole POLSUS.

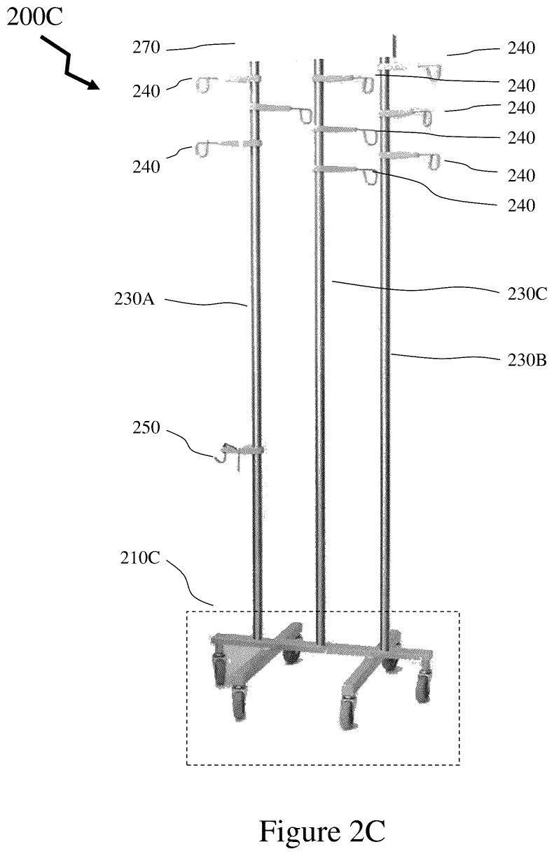

FIGS. 3A and 3B depict base configurations for POLSUS configurations according to embodiments of the invention. Referring to FIGS. 3A and 3B there depicted plan and bottom views 300A and 300B respectively for a mobile base such as mobile POLSUS bases 210A, 210B or 210C in FIGS. 2A to 2C respectively or as the bases 730, 830 and 930 in FIGS. 7 to 9 respectively. The base in plan and bottom views 300A and 300B is depicted in triple pole configuration where three poles 360 are depicted upon the cross-bar 310 and attached via first bolts 360. Also attached to the cross-bar 310 are a pair of bars 320A and 320B which are joined to the cross-bar 310 by second bolts 350 and nuts 370. Attached to the base are first to sixth wheels 340A to 340F wherein the third and sixth wheels 340C and 340G are disposed at either end of the cross-bar. First and fifth wheels 340A and 340E are attached to the first bar 320A and the second and fourth wheels 340B and 340D are attached to the second bar 320B.

The first to sixth wheels 340A to 340F may be casters, wheels, swivel casters, locking casters and locking swivel casters for example. The cross-bar 310 supports configurations with one, two or three poles 330 as depicted in FIGS. 7 to 9 respectively. Accordingly, the base depicted in FIGS. 3A and 3B respectively supports a wide range of POLSUS configurations based upon one, two and three pole variants and the multiple different pole supports etc. which can be attached to them. As evident from FIG. 3B the first to sixth wheels 340A to 340F are disposed upon a circle.

Within other embodiments of the invention the first to sixth wheels 340A to 340F may be replaced by sliders or other attachments which allow low friction movement of the mobile base upon a floor. Alternatively, within other embodiments of the invention the first to sixth wheels 340A to 340F may be replaced with feet such that the base provides a free-standing non-mobile support in conjunction with the poles, pole supports etc.

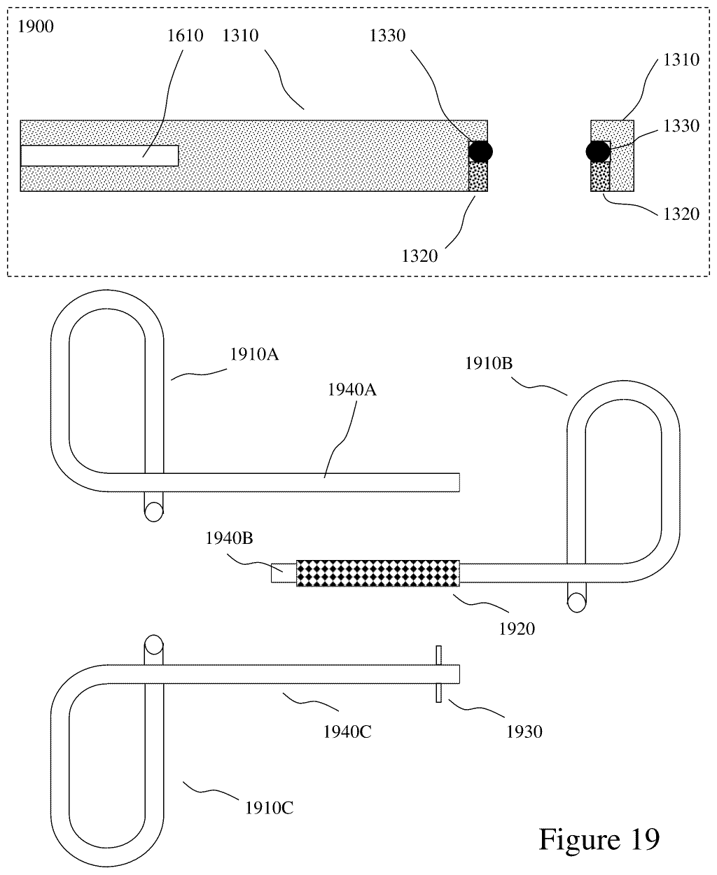

Now referring to FIG. 4 there is depicted an exemplary POLSUS 400 according to embodiments of the invention employing a triple pole configuration with first to third poles 230A, 230B and 230C attached to the base 430. Disposed at the top is a Joining Element 270 which prevents the first to third poles 230A, 230B and 230C flexing in different directions as the POLSUS 400 is moved. Disposed at the top are a plurality of FLSH pole supports 410 before being moved into their use positions, a hook pole support 250 and a Power Bar Shield 420. The Power Bar Shield 420 as described and depicted below in respect of FIG. 18 whilst allowing elements to be attached to pole supports which require electrical power such as monitors, pumps, infusion pumps, CPAP machines, etc. through a power bar is intended to mitigate fluid drips, spills, leaks, etc. from elements attached above the power bar. Optionally, the Power Bar Shield 420 allows protection of equipment attached to the POLSUS. Within embodiments of the invention the Power Bar Shield 420 has mounting features for the attachment of a power bar such that the power bar can be installed/replaced without affecting the Power Bar Shield 420. Within other embodiments of the invention the Power Bar Shield 420 and power bar may be an integrated assembly such that replacement of the power bar also replaces the Power Bar Shield 420.

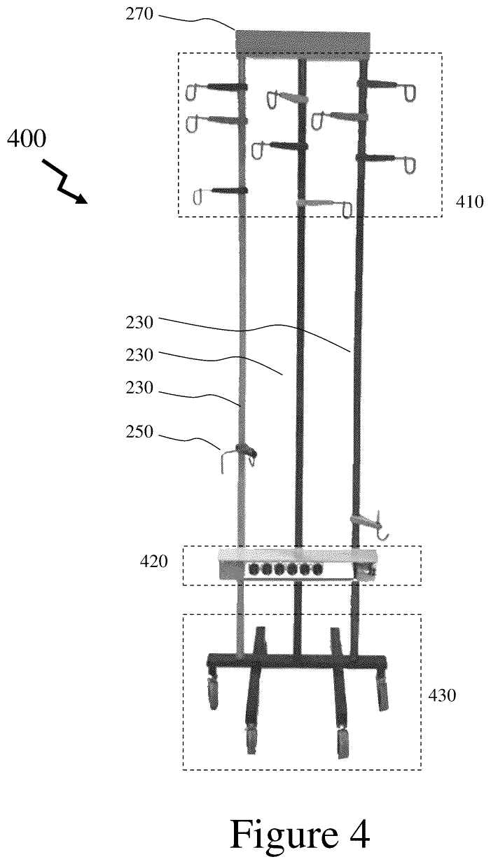

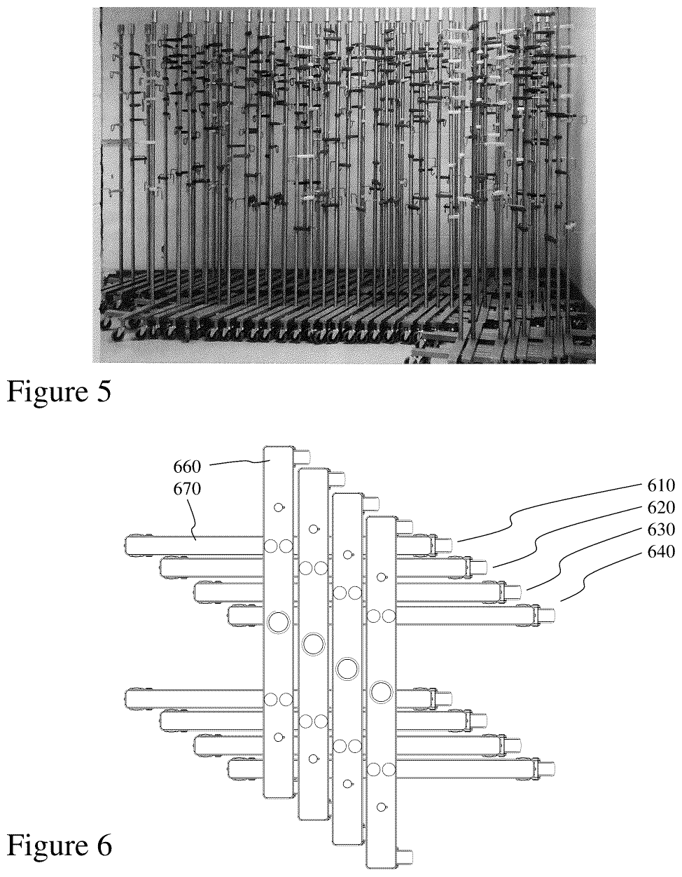

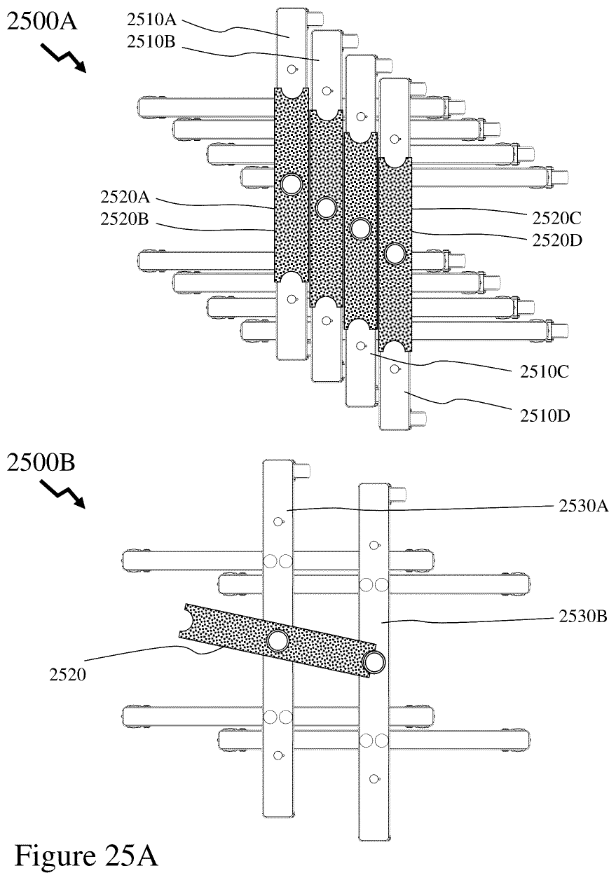

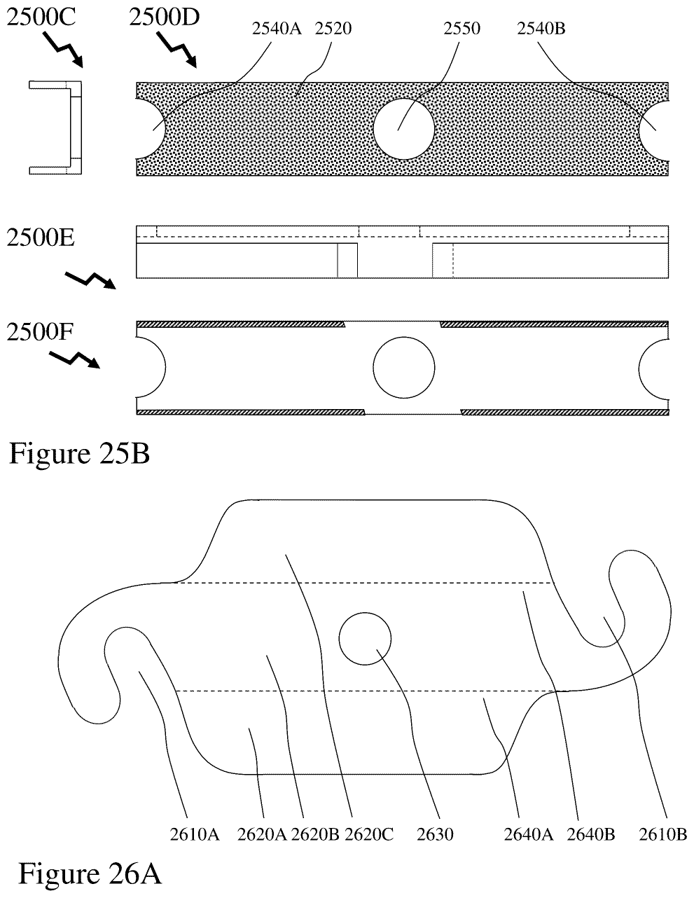

Referring to FIG. 5 depicts an exemplary demonstration of compact high density storage of POLSUS configurations according to embodiments of the invention exploiting a base such as depicted in FIGS. 3A and 3B respectively. Within FIG. 5 three banks of POLSUS are depicted from the front to the back. The front bank comprising 8 POLSUS units, the second rank comprising 30 POLSUS units and third rank comprising 35 POLSUS units. As evident the POLSUS can be stored with minimal footprint. This is achieved as depicted in FIG. 6 where 4 POLSUS 610 to 640 are shown nested together. Accordingly, this is achieved by establishing the upper height of the pair of bars 670 as lower than that beneath the cross-bar 660 such that the bars 670 of other bases fit beneath each cross-bar, for example parallel to each other for increased nesting of bases. Optionally, a buffer may be attached to the base and/or post(s) mounted onto the base such that if the base is brought into position with respect to another base then any elements attached to the post(s) such as IV drips, diffusion pumps, infusion pumps etc. do not hit others on the other base. Such elements, which the inventors refer to as anti-nesters being depicted and described below with respect to FIGS. 25A to 26B respectively. Alternatively, the minimum distance between two bases may be established in dependence upon buffers mounted to each base.

Now referring to FIG. 7 there is depicted a perspective view of an exemplary modular POLSUS 700 providing a single pole POLSUS configuration according to an embodiment of the invention. As depicted the POLSUS 700 comprises a base 730 such as described and depicted above with respect to FIGS. 3A and 3B respectively. A single pole 710 is attached to the base 730 and is equipped with a cap element 720. The cross-bar 740 of the base 730 having first and second locations 750A and 750C for subsequent attachment of other poles in some embodiments of the invention or allowing the same base 730 to be employed in provisioning double and triple pole POLSUS 800 and 900 such as described and depicted with respect to FIGS. 8 and 9 below.

Referring to FIG. 8 there is depicted a perspective view of an exemplary modular POLSUS 800 providing a double pole POLSUS configuration according to an embodiment of the invention. As depicted the POLSUS 800 comprises a base 830 such as described and depicted above with respect to FIGS. 3A and 3B respectively. A pair of poles 810A and 810B are attached to the base 830 and are equipped with cap elements 820A and 820B respectively which may be replaced with a Joining Element 270. The cross-bar 840 of the base 830 having a location 850 for subsequent attachment of another pole in some embodiments of the invention or allowing the same base 830 to be employed in provisioning single and triple pole POLSUS 700 and 900 such as described and depicted with respect to FIGS. 7 above and 9 below.

Now referring to FIG. 9 there is depicted a perspective view of an exemplary modular POLSUS 900 providing a triple pole POLSUS configuration according to an embodiment of the invention. As depicted the POLSUS 900 comprises a base 930 such as described and depicted above with respect to FIGS. 3A and 3B respectively. A set of poles 910A, 910B and 910C are attached to the base 930 and are equipped with cap elements 920A, 920B and 920C respectively which may be replaced with a Joining Element 270. Accordingly, by removal of one or two poles the same base 930 can be employed in provisioning single and triple pole POLSUS 700 and 800 such as described and depicted with respect to FIGS. 7 and 8 below.

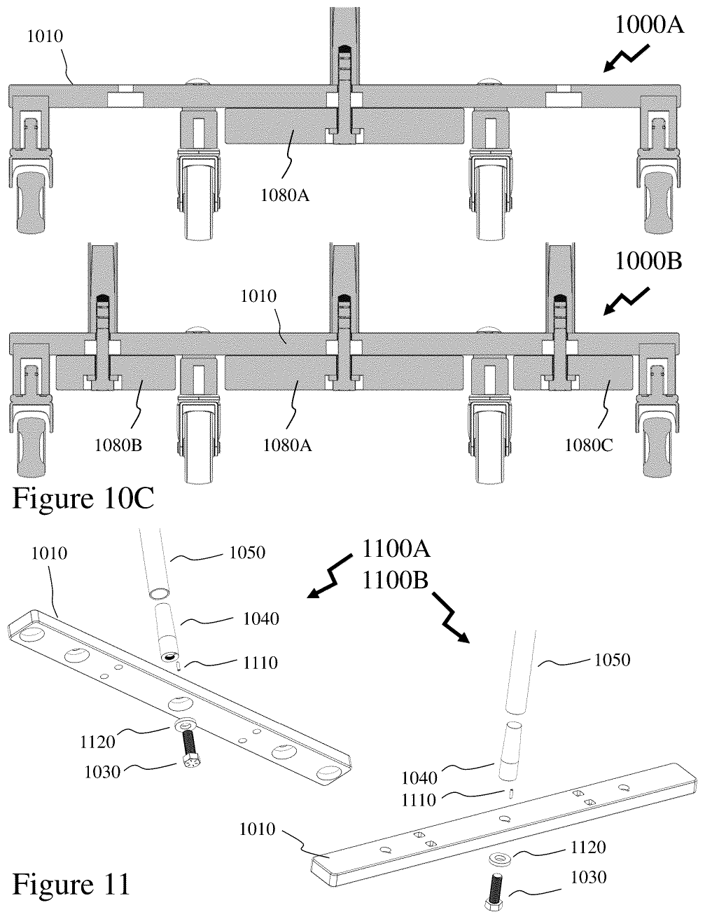

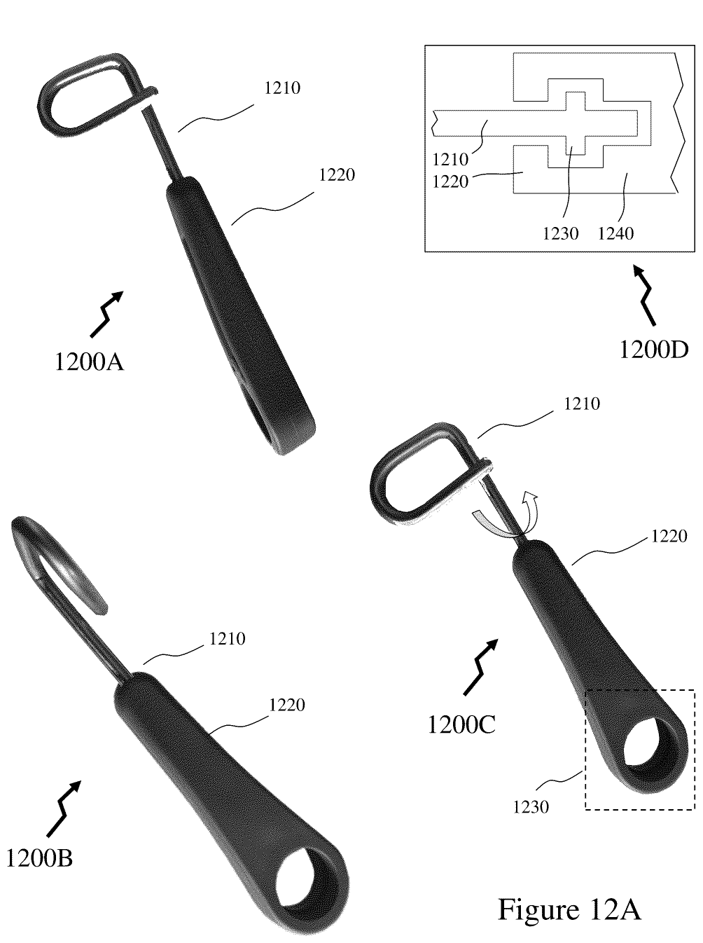

Referring to FIG. 10A there is depicted a cross-sectional elevation view of an exemplary modular POLSUS system providing configurable POLSUS systems according to embodiments of the invention such as described and depicted above in respect of FIGS. 2A to 9 respectively. As depicted the cross-section is through along the longitudinal axis of a cross-bar 1010, such as cross-bar 310 in FIGS. 3A and 3B. Accordingly, there are depicted first and fourth wheels 1070A and 1070D at either end of the cross-bar 1010. Also depicted are the pair of bars 1060A and 1060B which are mounted orthogonally to the cross-bar 1010 which have at either end a wheel, wherein those depicted are second and third wheels 1070B and 1070C respectively. The openings 1020 within the cross-bar 1010 allow the cross-bar 1010 to have additional poles mounted to it in the same manner as the pole 1050 is depicted in FIG. 10 and in FIG. 11. As depicted in these Figures the pole 1050 has an insert 1040 at the bottom end which is an interference fit such that when inserted the insert 1040 is retained within the pole 1050 by mechanical friction, pressure etc. The insert 1040 has a threaded insert which mates to a bolt 1030 which goes through an opening 1020.

Referring to FIG. 10B there is depicted a cross-sectional elevation view of an exemplary modular POLSUS system similar cross-section as depicted in FIG. 10A with the cross-section is through along the longitudinal axis of a cross-bar 1010B, such as cross-bar 310 in FIGS. 3A and 3B. As evident in FIG. 10B the cross-bar 1010B is now thicker than the cross-bar 1010 in FIG. 10A. Accordingly, these cross-bars 1010B are heavier than cross-bars 1010. Accordingly, it would be evident that a POLSUS employing cross-bars 1010B can support increased weight and/or higher weight placement.

Referring to FIG. 10C there are depicted first and second POLSUS 1000A and 1000B respectively comprising single and triple pole configurations respectively. Each of first and second POLSUS 1000A and 1000B employ cross-bars 1010 in common with the POLSUS depicted in FIG. 10A. However, as evident in first image 1000A beneath the single pole a first weight 1080A has been added which is attached when the pole is attached, simply with a longer bolt than in FIG. 10A. Accordingly, first weight 1080A provides additional weight to a POLSUS according to an embodiment of the invention. Within an embodiment of the invention the first weight 1080A may have the same width as that of cross-bar 1010 so that no additional surfaces for collecting dust, debris, fluids etc. exist although within other embodiments of the invention the first weight 1080A may be wider than cross-bar 1010 and optionally profiled to minimize retention of dust, debris, fluids etc. Optionally, first weight 1080A may be formed from the same material as the cross-bar 1010, e.g. coated steel, stainless steel, aluminum, plastic, etc. or it may be a different material, e.g. coated steel, stainless steel, coated lead etc. The assembly of an additional weight at initial construction of the POLSUS removes the requirement for additional mounting holes etc. which impact the ability of maintaining a clean disinfected POLSUS in medical applications, for example.

Now referring to second image 1000B beneath the central pole is first weight 1080A has been added which is attached when the pole is attached, simply with a longer bolt than in FIG. 10A. The other poles also have second and third weights 1080B and 1080C disposed beneath them which are attached in a similar manner as first weight 1080A. Accordingly, first to third weights 1080A to 1080C respectively provide additional weight to a POLSUS according to an embodiment of the invention. Within an embodiment of the invention the first to third weights 1080A to 1080C respectively may have the same width as that of cross-bar 1010 so that no additional surfaces for collecting dust, debris, fluids etc. exist although within other embodiments of the invention the first to third weights 1080A to 1080C respectively may be wider than cross-bar 1010 and optionally profiled to minimize retention of dust, debris, fluids etc. Optionally, first to third weights 1080A to 1080C respectively may be formed from the same material as the cross-bar 1010, e.g. coated steel, stainless steel, aluminum, plastic, etc. or they may be formed from different materials, e.g. coated steel, stainless steel, coated lead etc. The assembly of an additional weight at initial construction of the POLSUS removes the requirement for additional mounting holes etc. which impact the ability of maintaining a clean disinfected POLSUS in medical applications, for example.

Cross-bar 1010 being, for example, cross-bar 310 in FIG. 3A or cross-bar 660 in FIG. 6 although it is noted that in this configuration the additional weights, such as one or more of first to third weights 1080A to 1080C, disposed beneath the cross-bar would prevent the nesting of POLSUS such as depicted in FIG. 6. Optionally, whilst also limiting the nesting capability additional weights could also be applied to the upper side of the bar, such as bars 670 in FIG. 6. However, if the additional weights were attached to the upper side of the cross-bar prior to attachment of the pole(s) then the nesting capability could be maintained. Similarly, if additional weights were attached to the underside of the bars attached to the cross-bar, such as bars 670 in FIG. 6, then the nesting capability of the POLSUS as depicted in FIG. 6 could also be maintained. Optionally, additional weights may be applied therefore to one or more of the upper side of the cross-bar, the underside of the cross-bar, upper side of one or more bars, and the underside of one or more bars depending upon whether a requirement exists for the POLSUS with such weights to have the nesting capability or not.

As depicted in FIG. 11 in first and second exploded schematics 1100A and 1100B the assembly of the pole 1050 to the cross-bar 1010 also includes a washer 1120, which may be omitted, and a rod 1110 which fits into a first hole within the bottom of the insert 1040 and a second hole within the cross-bar 1010 such that the rotational position of the pole 1050 with the interference fitted insert 1040 is fixed relative to the cross-bar 1010 once everything is assembled.

Within other embodiments of the invention the pole 1050 and insert 1040 may be a single piece-part or pole 1050 may be solid with the threaded insert and the first hole formed within an end or optionally both ends of the pole 1050. Optionally, a tubular pole 1050 may have a second insert 1040 at the other end such that a cap may be screwed onto the end of the pole 1050. Optionally, the other end of the pole from that attached to the cross-bar 1010 may have an insert with no threaded insert or first opening to seal it.

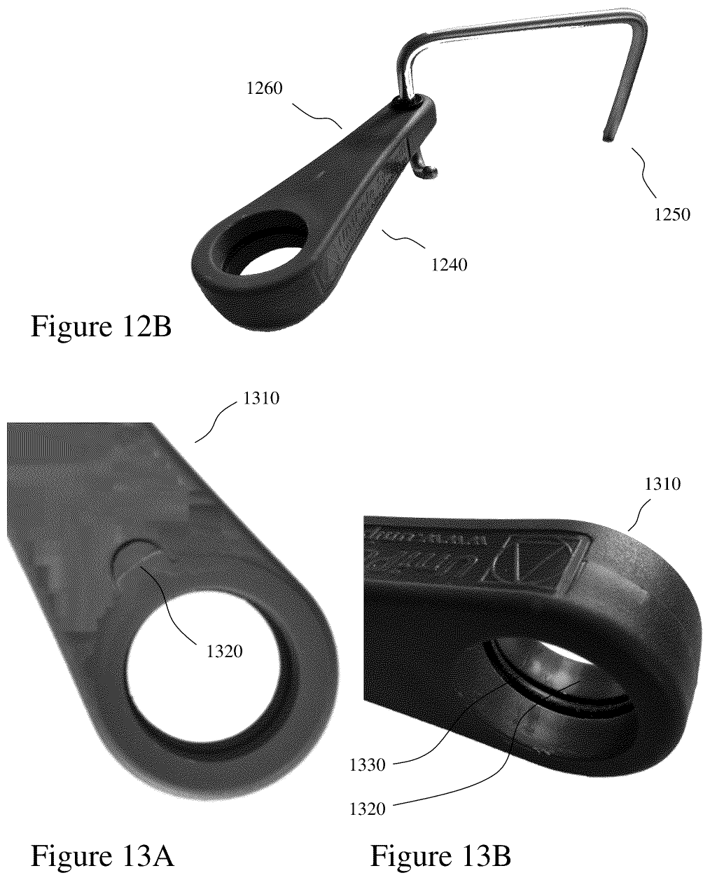

FIG. 12A depicts an exemplary FLSH pole support providing a tube retainer such as described above with FLSH pole support 240 in FIGS. 2A to 2C respectively according to an embodiment of the invention for attachment to a POLSUS configuration according to embodiments of the invention. In first image 1200A the FLSH pole support is depicted in a side perspective view and comprises a Support Body 1220 with FLSH Fitting 1210. Second and third images 1200B and 1200C depict the FLSH pole support in bottom perspective view and top perspective view respectively. In third image 1200C the FLSH Fitting 1210 is rotated relative to the Support Body 1220 as depicted in first and second images 1200A and 1200B respectively. As evident from third Image 1200C the FLSH Fitting 1210 may rotate relative to the Support Body 1220.

Also depicted in third image 1200C is Pole Fitting 1230. Within embodiments of the invention the FLSH Fitting 1210 may be retained within the Support Body 1220 through an interference fit of the FLSH Fitting 1210 within a hole within the end of the Support Body 1220, the end of the FLSH Fitting 1210 may be knurled or have additional surface finishes to enhance retention when fitted into the hole within the end of the Support Body 1220 of the Support Body 1220 is molded around the FLSH Fitting 1210. Optionally, as depicted in insert 1200D if the Support Body 1220 is molded around the FLSH Fitting 1210 then the FLSH Fitting 1210 may comprise a Projection 1230, e.g. a pair of rod like sectional projections or a ring, which is retained within a Recess 1240 within the Support Body 1220 to prevent the FLSH Fitting 1210 being pulled out from the Support Body 1220. Within embodiments of the invention the Projection 1230 and Recess 1240 may lock the orientation of the FLSH Fitting 1210 relative to the Support Body 1220 or the FLSH Fitting 1210 may freely rotate relative to the Support Body 1220.

According to embodiments of the invention the FLSH Fitting 1210 may be replaced with a hook element to provide the hook pole support 250 as depicted in FIGS. 2A to 2C respectively or with other elements to provide different pole supports. Accordingly, a common Support Body 1220 may be employed with multiple fittings within some embodiments of the invention whereas in others different support bodies may be employed although the Pole Fitting 1230 may be common to all. Although it would be evident that the Pole Fitting 1230 may have different dimensions to support different diameter poles, such as Pole 230 or first to third Poles 230A to 230C respectively for example.

Now referring to FIG. 12B there is depicted a hook pole support such as hook pole support 250 depicted in FIGS. 2A to 2C respectively. As depicted the hook pole support comprises a support body 1240 and hook element 1250. The body of the hook support depicted in FIG. 12B is similar to that of support body 1210 except at the distal end from the mounting to the post wherein the fitting now fits into a hole through the support body 1240 rather than into the end of the support body as depicted in FIG. 12A. The hook element 1250 is inserted through a grommet 1260 which is fitted to the hole through the support body 1240.

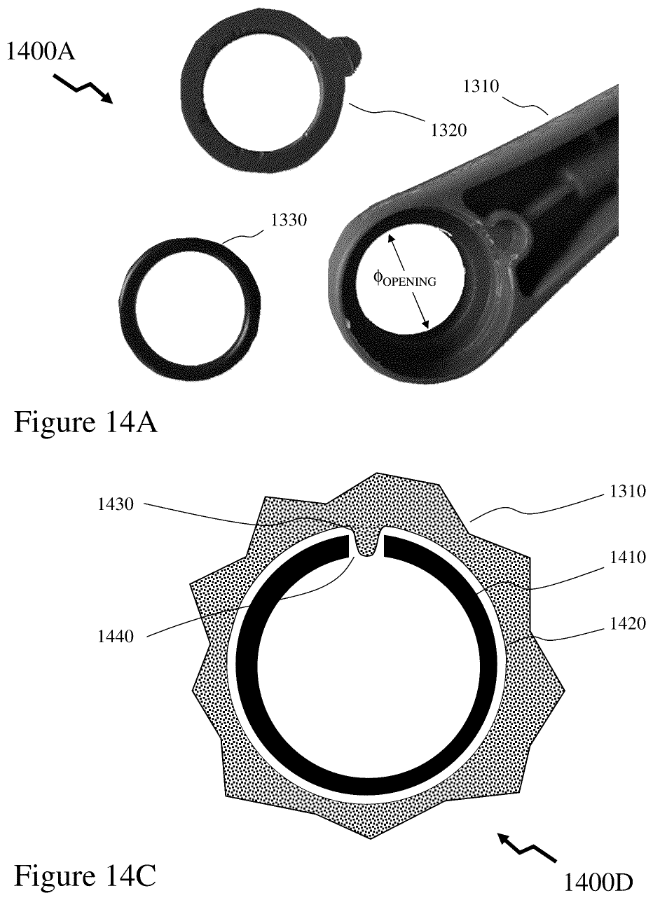

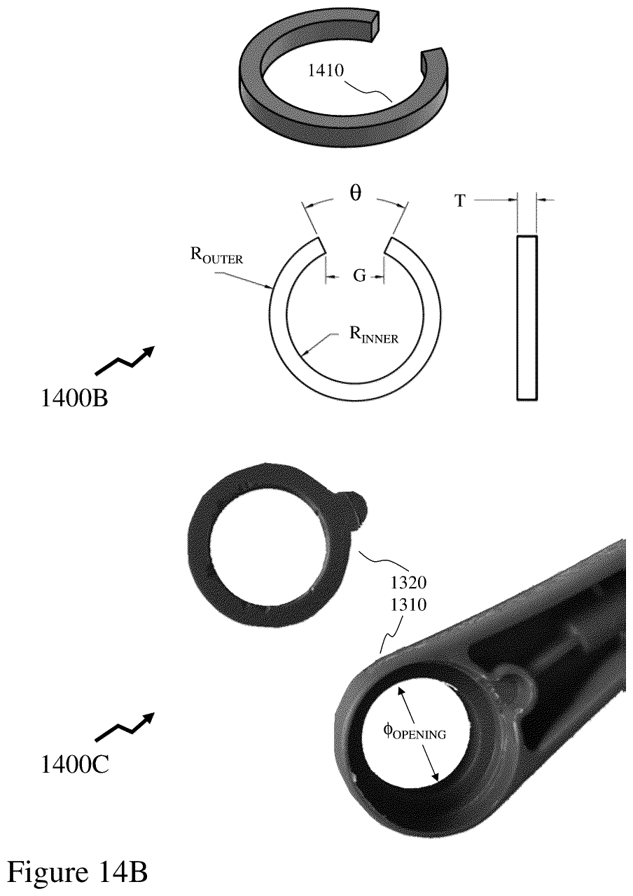

Referring to FIGS. 13A-13B and 14A-14B respectively there are depicted close-up and assembly images of the Pole Fitting 1230 of the Support Body 1220. As depicted in FIG. 13A the support body comprises a Shell 1310 and Insert 1320. In FIG. 13B the Shell 1310 and Insert 1320 are depicted together with O-ring 1330 which is disposed between the Insert 1320 and Shell 1310. In FIG. 14A an exploded assembly 1400A of Pole Fitting 1230 of the Support Body 1220 comprising the Shell 1310, Insert 1320 and O-ring 1330 which are depicted individually. Within an embodiment of the invention the Shell 1310 and Insert 1320 may be formed as separate piece parts and assembled with the O-ring 1330. Alternatively, the Shell 1310 and Insert 1320 may be molded as a single piece part with the O-ring 1330 disposed within the mold(s) such that there is no need for subsequent assembly.

Whilst the pole support with an O-ring 1330 performs its desired function in some embodiments of the invention, such as medical clinics, hospitals etc. the increasingly aggressive cleaning solutions being employed, for example as a result of COVID-19, may in some instances attack and/or degrade the material(s) employed for the O-ring 1330. Accordingly, as depicted and described below in respect of FIG. 14B the inventor has replaced the O-ring 1330 with a Split Ring 1410. In common with the O-ring 1330 the Split Ring 1410 “floats” within a cavity or pocket formed between the Shell 1310 and Insert 1320. Accordingly, Split Ring 1410 may be formed from a polymer, for example, such as a thermoplastic, a thermosetting polymer, an elastomer or a combination thereof. For embodiments of the invention providing enhanced chemical resistance the polymer, for example, is chemically resistant to a wide range of chemical cleaning agents. As evident in FIG. 14B in first and second images 1400B and 1400C respectively the Split Ring 1410 whilst shaped like a spring clip it is not designed to hold one part against another as it is “sandwiched” between two surfaces to create a sliding resistance, thereby serving its gripping purpose with improved chemical resistance.

FIG. 14B depicts first and second images 1400B and 1400C respectively representing an exploded assembly image for an exemplary pole support according to an embodiment of the invention. As depicted the Pole Fitting 1230 of the Support Body 1220 is depicted comprising the Shell 1310 and Insert 1320 but not the O-ring 1330 depicted in FIG. 14A has been replaced with a Split Ring 1410. As depicted the Split Ring 1410 is formed from a circular element with an inner radius, R_{INNER}, an outer radius R_{OUTER} with a thickness, T, having a gap of angular extent, θ, with a minimum distance, G, at the inner edge of the Split Ring 1410. Within an embodiment of the invention the Shell 1310 and Insert 1320 may be formed as separate piece parts and assembled with the Split Ring 1410. Alternatively, the Shell 1310 and Insert 1320 may be molded as a single piece part with the Split Ring 1410 disposed within the mold(s) such that there is no need for subsequent assembly.

Now referring to FIG. 14C there is depicted an image 1400D of a variant assembly as depicted in FIG. 14B in first and second images 1400B and 1400C respectively wherein the inner Periphery 1420 of the opening within the Shell 1310 has a Projection 1430 which engages the Opening 1440 within the Split Ring 1410 thereby preventing rotation of the Split Ring 1410 relative to the Shell 1310.

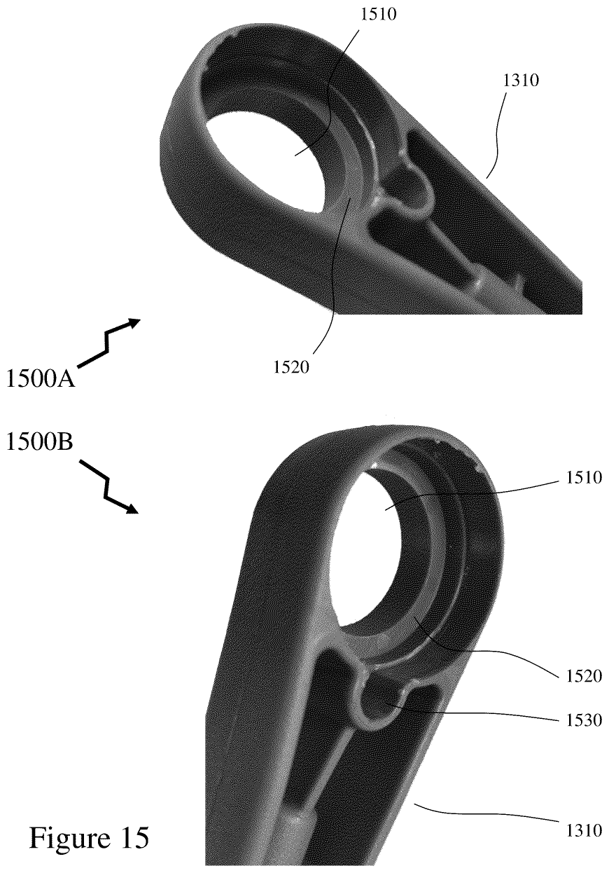

Now referring to FIG. 15 there are depicted first and second detailed images 1500A and 1500B respectively of the Shell 1310, which forms the body portion (Support Body 1220) of an exemplary pole support according to the embodiments of the invention, as depicted in FIGS. 12 to 14 respectively. Accordingly, the Shell 1310 at the end forming the pole fitting portion of the support body has a first diameter region 1510 and a second diameter region 1520. The O-ring, for example O-ring 1330 in FIG. 14, fits into the second diameter region 1520 whilst the inert, for example Insert 1320 in FIG. 14, fits into the first diameter region 1510 thereby sandwiching the O-ring into position. Optionally, within other embodiments of the invention the Shell 1310 and Insert 1320 may be formed as a single piece into which there is formed a recess within which the O-ring is inserted. Optionally, the Shell 1310 at the end forming the pole fitting portion rather than a circular design has a generally circular design with feature 1530. The feature 1530 matching another feature upon the insert, for example Insert 1320 in FIG. 14, such that the insert does not rotate once inserted into the opening of the Shell 1310.

Within embodiments of the invention the Insert 1320 is retained in position within the Shell 1310 by a material such as an adhesive, glue, elastomer etc. or it may be retained in position through an interference fit between the outside of the Insert 1320 and the interior of the opening within the Shell 1310. Alternatively, the Insert 1320 may be ultrasonically welded into position within the Shell 1310.

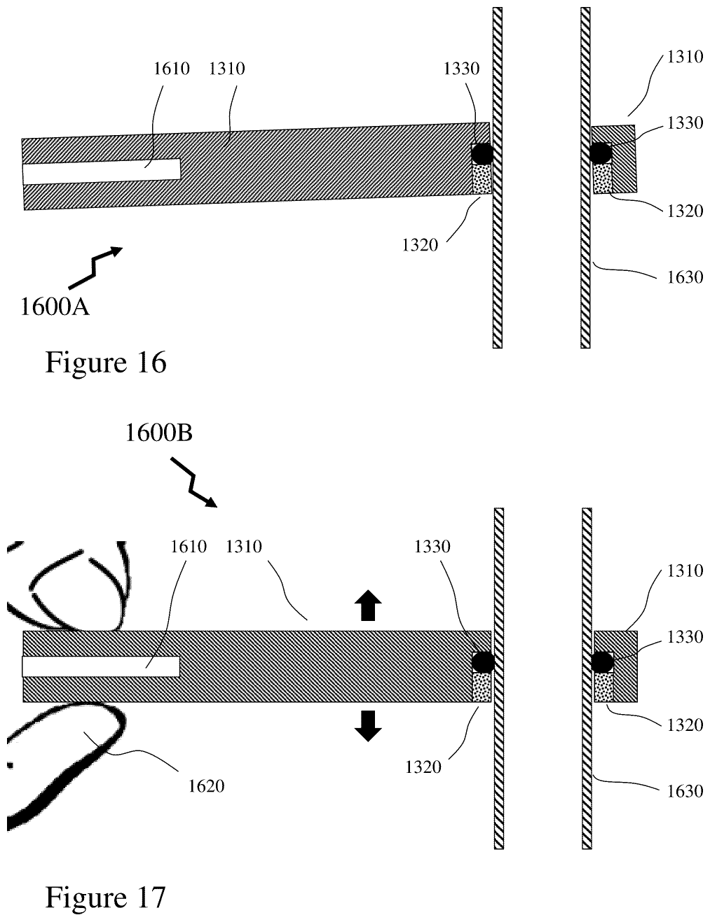

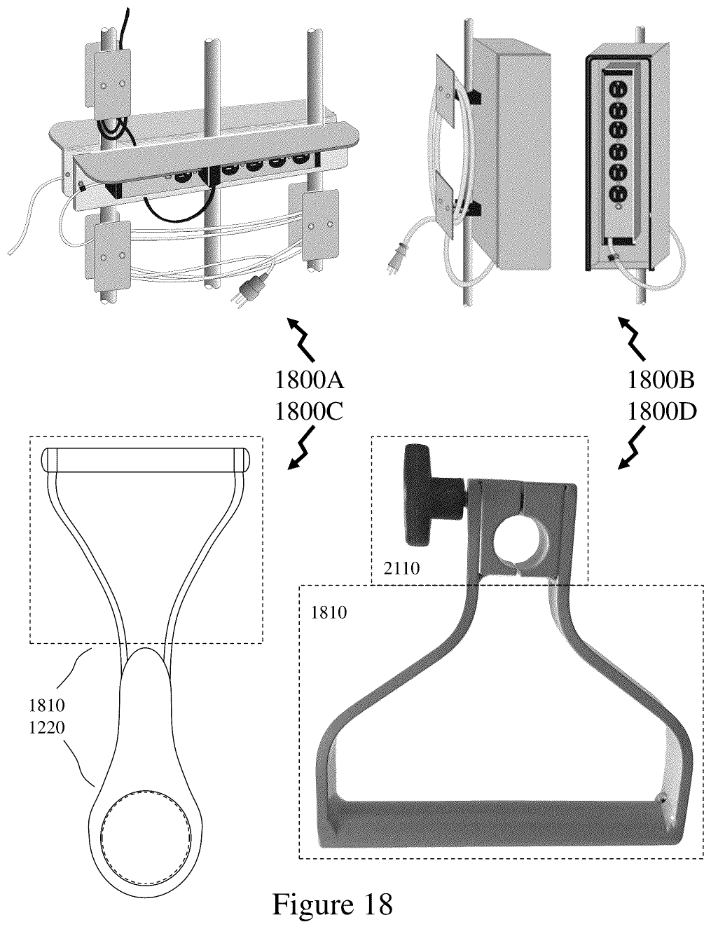

Now referring to FIGS. 16 and 17 there are depicted first and second images 1600A and 1600B respectively for an exemplary pole support according to the embodiments of the invention as depicted in FIGS. 2A to 15 respectively in movable and locked configurations respectively. In first image 1600A a pole support, for example FLSH pole support 240 in FIGS. 2A to 2C, is depicted in deployed position in cross-section showing the pole support comprising Shell 1310, Insert 1320, and O-ring 1330. Accordingly, the pole support is cantilevered down through the weight of the Shell 1310 projecting away from the post 1630 such that the Shell 1310 in conjunction with the O-ring 1330 retain the pole support in place upon the post 1630. It would be evident that the provisioning of any additional loading at the distal end of the Shell 1310 away from the post 1630 further cantilevers the Shell 1310 down when not manually lifted so that the Shell 1310 engages against the post 1630 and is retained in position absent any additional action from the user. Accordingly, the pole support employing Shell 1310, Insert 1320 and O-ring 1330 may be manually positioned on the post 1630 with one hand of a user, e.g. a nurse, medical technician, etc.), allowing them to be holding an item in their other hand and the pole support remains in position when the user releases it. This is enabled by the cantilever principle of the design and whilst moving the pole support the cantilever is inactive.

In second image 1600B a user lifts the Shell 1310 into a horizontal position relative to the post 1630 allowing the Shell 1310, and therein the post support, to be moved up or down on the post. The user can therefore move the post support with one hand allowing them to maintain their other hand free to hold a wheelchair, IV bag, etc. The inventor has established that the appropriate relative dimensions of the opening within the shell portion of the post support, diameter ϕ_{OPENING}, and the diameter of the post in conjunction with the O-ring upon which it or they are mounted allow for retention of the post support upon the post without any other means and allow for a low impact lifting of the distal end of the post support from the post for a user to move the post support. These appropriate relative dimensions also being dependent upon a height of the support along the axis of the post upon which it mounts. In contrast, prior art supports for posts employ screws, clamps, or other retention means to maintain the post support in position and must be undone/done up to move them. Accordingly, their movement and positioning is a dedicated task for a user requiring both hands.

In each of first and second images 1600A and 1600B the distal end of the Shell 1310 from the post 1630 has an opening 1610 formed within it. Accordingly, inserted into the opening 1610 is a fitting which defines the function of the post support. For example, a curved loop with a straight portion for insertion into the opening 1610 may provide the FLSH fitting for a FLSH post support and a rod with a bend may form the hook portion of a hook post support. Whilst in embodiments of the invention the post support is sold, distributed and employed as a discrete piece part within other embodiments of the invention the post support may be sold as a set of supports to which a user adds the appropriate fitting or a fitting may be changed/replaced subsequently at a later point in time. Within other embodiments of the invention the Shell 1310 may be molded onto a fitting which has features to retain the fitting in place such that it cannot be removed/replaced etc.

FIG. 18 depicts additional equipment for use in conjunction with POLSUS according to embodiments of the invention. In first image 1800A a power bar shield such as Power Bar Shield 420 in FIG. 4 is depicted for use in conjunction with a horizontally orientated electrical distribution panel for attachment to a double pole POLSUS such as depicted in FIGS. 2B and 8 respectively or a triple pole POLSUS such as depicted in FIGS. 2C and 9 respectively is depicted. The power bar shield preventing a direct path of fluid drips, fluid leaks, fluid spills etc. from elements attached to the POLSUS above the power bar onto the power bar.

In second image 1800B there are depicted front and rear images of a power bar shield for use in conjunction with a vertically orientated electrical distribution panel for attachment to a pole of a POLSUS. Again the power bar shield being to prevent fluid drips, fluid leaks, fluid spills etc. from elements attached to the POLSUS above the power bar impinging directly onto the power bar. It would be evident that as depicted in FIG. 4 a single Power Bar Shield 420 may be attached on one side of the POLSUS or that a pair may be employed as depicted in first image 1800A in conjunction with power bars on either side of the POLSUS.

Also depicted in first and second images 1800A and 1800B are cable management elements which attached to the poles of the POLSUS. It would be evident that the power bar shield can be employed with more than one outlet power bar and that different manufacturers power bars can be attached making the power bar shield agnostic to the actual power bar attached. Further, multiple Power Bar Shields 420 on one or both sides of a POLSUS.

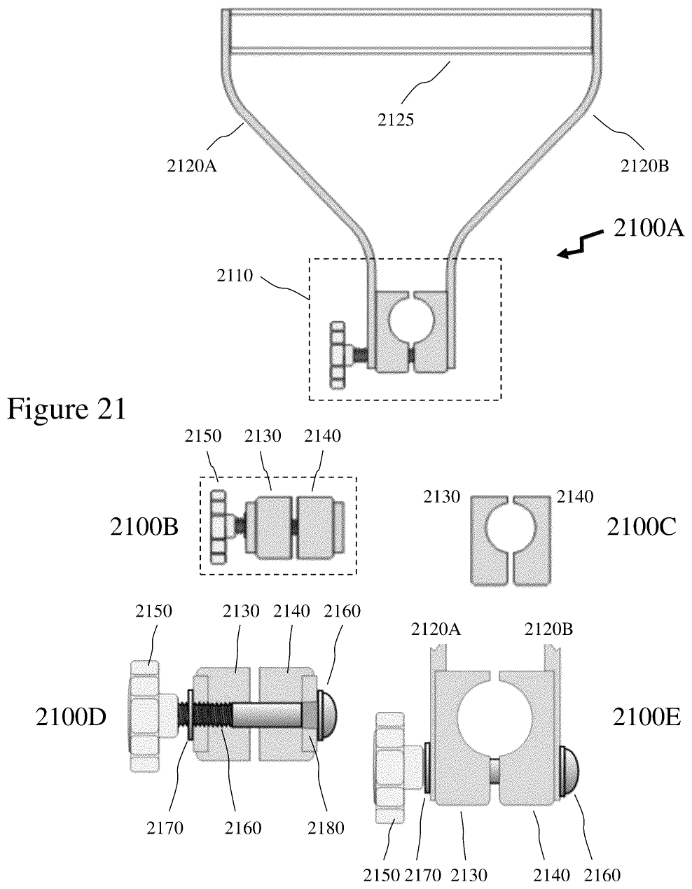

In third image 1800C there is depicted a handle for attachment to a pole allowing a user to move the POLSUS where the Handle 1810 is attached via a Support Body 1220 as described and depicted with respect to FIGS. 12A to 17 respectively. Fourth image 1800D depicts another handle for attachment to a pole allowing a user to move the POLSUS comprising a Handle 1810 which attaches to a pole via a Connection Hub 2110 as described and depicted below in respect of FIG. 21.

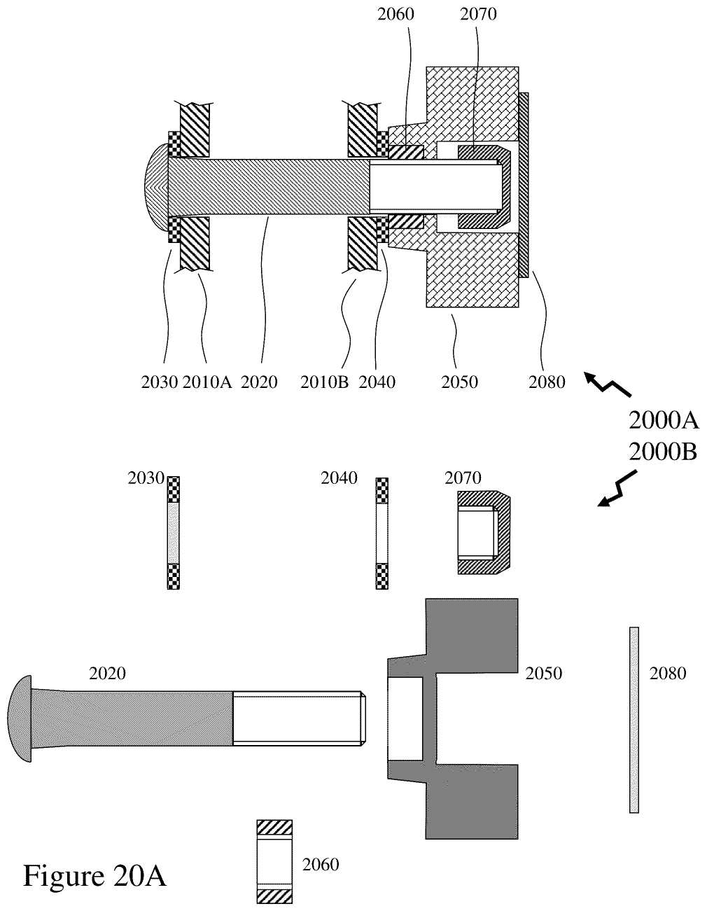

Referring to FIG. 19 there is depicted a Hanger Element 1900 such as described and depicted in FIG. 16 comprising the Shell 1310, Insert 1320, O-ring 1330 and opening 1610 (e.g. a circular bore). Referring to first Hanger Arm 1910A, of the cable/tube management (FLSH) pole support type as described and depicted in FIGS. 2A to 2C with cable/tube management (FLSH) pole supports 240, is depicted. The linear portion 1940A being dimensioned for an interference fit within the opening 1610 such that it is retained and requires a predetermined force to remove it from the opening 1610 wherein it will not come out during normal use, for example. In such a configuration the pole support arm may be of varying design and assembled with a common shell and replaced by applying sufficient force to withdraw the liner portion 1940A. This may be prior to shipping or upon their receipt by a customer.

Second Hanger Arm 1910B in contrast exploits a linear portion 1940B which has a diamond knurled exterior region 1920 such that the required force to remove the linear portion 1940B once inserted into the opening 1610 is such that replacement options are not viable. Accordingly, the hanger arms with knurled exterior regions would be pre-assembled prior to shipment to a customer. Third Hanger Arm 1910C exploits a linear portion 1940C with a rod 1930. In this configuration the Shell 1310 of the pole support would be molded around the linear portion 1940C and rod 1930.

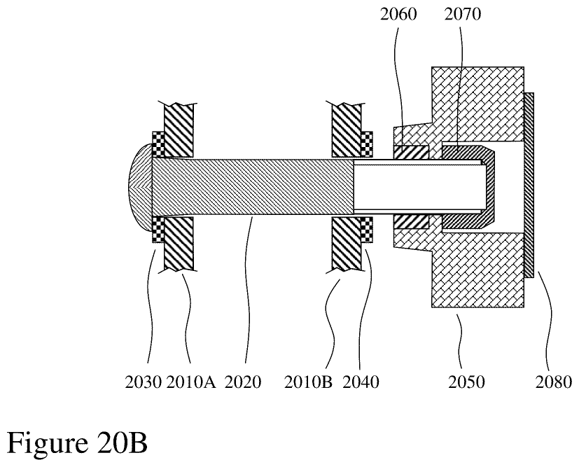

FIG. 20 depicts an adjustable resistance knob with retaining mechanism according to an embodiment of the invention. It would be evident to one of skill in the art that there are instances where an adjustable hub, handle or bracket is required to be mounted to a POLSUS. This may, for example, be to mount a pump, distribution board, monitor or other item of heavy equipment beyond the capabilities of the pole supports described and depicted in FIGS. 12A to 16 respectively. Alternatively, a handle which is fixed in position may be required. However, whilst prior art mounting solutions exist exploiting a knob allowing a user to manually do up the clamping fixture in position; these have two limitations. Firstly, this knob can work loose through vibration. Secondly, the knob comes out even against prior art attempts to secure them such that the knobs become detached, lost etc.

Accordingly, the inventor has addressed this with the adjustable resistance knob described and depicted in respect of FIGS. 20A and 20B respectively. The design shown below in FIGS. 20A and 20B addresses the problem of knob loss in a manner which is enhanced and economically beneficial relative to prior art solutions which require modification of the threaded stem tip of a knob after the knob has been installed. Beneficially embodiments of the invention allow a single knob to be employed on any length of stem of same thread whilst the design principles are easily adapted to stems of different diameters, thread, left versus right hand thread etc.

First image 2000A in FIG. 20A depicts the adjustable resistance knob in a clamped condition whilst FIG. 20B depicts the adjustable resistance knob in an unclamped condition. Second image 2000B in FIG. 20B depicts the elements of the adjustable resistance knob according to an embodiment of the invention in exploded form. A Bolt 2020 is stabilized between the two opposing faces, first Face 2010A and second Face 2010B which are compressed against when the Knob 2050 is turned clockwise (for a right handed thread) or anticlockwise (for a left handed threaded). Within the following description the Bolt 2020 is considered right hand threaded. However, one of skill would understand the other instance of left handed thread. When the Bolt 2020 is a carriage bolt it would be evident that providing a square opening within the first Face 2010A prevents rotation of the Bolt 2020 relative to the first Face 2010 as the Knob 2050 is turned clockwise or counterclockwise.

When the Knob 2050 is turned counterclockwise the first Face 2010A and second Face 2010B are decompressed such that the assembly can be moved but the knob cannot be rotated off the shaft because it is restricted. First Face 2010A and second Face 2020 being surfaces to engage against a fitting upon or around the pole, where the fitting and pole are not depicted for clarity. At the non-threaded end of the Bolt 2020 against first Face 2010A a first Washer 2030 is disposed. At the threaded end of the Bolt 2020 against second Face 2010B a second Washer 2040 is disposed. Disposed and retained within the end of the Knob 2050 towards the second Face 2010B is a Nut 2060 allowing the Knob 2050 to rotate on the thread of the Bolt 2020. Disposed within a distal end of the Knob 2050 away from the second Face 2010B is a cavity within which a Locknut 2070 is disposed upon the end of the thread of Bolt 2020. Accordingly, when the Knob 2050 is rotated counterclockwise the Knob 2050 moves along the thread of the Bolt 2020 towards the Locknut 2070. When the body of the Knob 2050 engages against the Locknut 2070 further rotation is prevented such that the Knob 2050 cannot be removed from the Bolt 2020. A Cover 2080 is disposed over the cavity within the Knob 2050 to prevent ingress of material affecting action of the Knob 2050. FIG. 20B depicts the Knob 2050 in the position that the body of the Knob 2050 has engaged against the Locknut 2070 thereby preventing further rotation of the Knob 2050 away from the second Face 2010B. Optionally, Nut 2060 may be a discrete element retained within the Knob 2050, a threaded insert retained within the Knob 2050, or a threaded portion of the Knob 2050.

Second image 2000B depicts in cross-section the elements of the adjustable resistance knob in exploded form, with first Face 2010A and second Face 2010B omitted for clarity, these comprising:
Bolt 2020;
First Washer 2030;
Second Washer 2040;
Knob 2050;
Nut 2060;
Locknut 2070; and
Cover 2080.

With respect to assembly of the adjustable resistance knob then an exemplary sequence comprises:
Installing the Bolt 2020 through the first Face 2010A and second Face 2010B in conjunction with first Washer 2030;
Place second Washer 2040;
Mount the Knob 2050 and tighten it to clamp the first Face 2010A and second Face 2010B against an object;
Add the Locknut 2070 and tighten to predetermined depth (e.g. with wrench or socket wrench); and
Fit the Cover 2080, for example this may be a snap fit to the opening within the Knob 2050 or attached with adhesive, epoxy, double-sided tape, cold-welded etc.

It would be evident to one of skill in the art that the length of the knob stem is contingent upon component need but that the design according to embodiments of the invention allows for alternative configurations, geometries, etc. such as a non-knob/thread stem combination (e.g. a lever) or a threaded rod inserted into a blind hole with a thread locker to substitute for a carriage bolt. Other variations would be evident to one of skill in the art.

Accordingly, referring to FIG. 21 there are depicted first to fifth images 2100A to 2100E of a Connection Hub 2110 discrete or configured for use with POLSUS according to embodiments of the invention, which may within some embodiments of the invention exploit the adjustable resistance knob as described and depicted in FIGS. 20A and 20B. Referring to first image 2100A the Connection Hub 2110 is depicted forming part of a handle for a POLSUS allowing a user to push or pull the POLSUS. The handle comprising a pair of Handle Shafts 2120A and 2120B and a Grip 2125. Accordingly, the Connection Hub 2110 with a removal resistant knob allows for securing the handle without driving a bolt into the surface of a pole.

In second image 2100B the Connection Hub 2110 elements of Knob 2150, first Clamp 2130 and second Clamp 2140 are depicted. It would be evident that the Connection Hub 2110 may be employed with non-tubular or non-circular elements by changing the first Clamp 2130 and second Clamp 2140. Within FIG. 21 first Clamp 2130 is functionally equivalent to first Face 2010A in FIGS. 20A and 20B respectively whilst second Clamp 2140 is functionally equivalent to second Face 2010B in FIGS. 20A and 20B respectively. These being depicted in isolation in third image 2100C.

Fourth image 2100D depicts a partial cross-section of the Connection Hub 2110 wherein it is depicted as comprising:
Knob 2150;
Washer 2170;
Bolt 2160;
First Clamp 2130;
Second Clamp 2140; and
Fitting 2180 within the second Clamp 2140 which retains the bolt such that it does not rotate when the Knob 2150 is rotated. For example, when the Bolt 2160 is a carriage bolt the Fitting 2180 is simply a square feature in the end of the second Clamp 2140. With other bolts other means of retaining them may be employed or these may be removed and an element allowing the other end of the Bolt 2160 from the Knob 2150 to be restrained, e.g. a handle, another knob fixed to the Bolt 2160; etc.

Fifth image 2100E depicts the Connection Hub 2110 with the pair of Handle Shafts 2120A and 2120B. Accordingly, there are depicted:
Knob 2150;
Washer 2170;
Bolt 2160;
First Clamp 2130; and
Second Clamp 2140.

It would be evident that the Connection Hub 2110 may be employed with other elements to provide a range of fittings for poles, bars, rods, etc. wherein the Connection Hub 2110 with a removal resistant knob allows for securing the handle without driving a bolt into the surface of a pole, bar, rod, etc. and preventing loss of the Knob 2150. Optionally, within other embodiments of the invention the Knob 2150 may be removable knob.

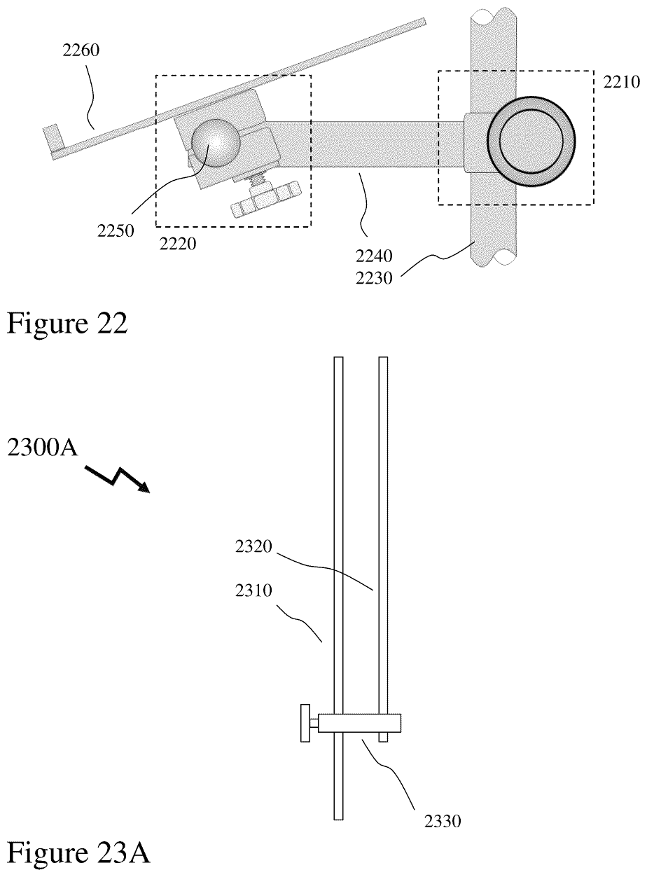

FIG. 22 depicts the Connection Hub 2110 of FIG. 21 for use with a POLSUS according to an embodiment of the invention for providing mountable/movable elements for a POLSUS, in this instance the element being adjustable platform. Accordingly, a first Hub 2210 allows an Arm 2240 to be attached to a Pole 2230. Disposed upon the Arm 2240 is a Mount 2250, e.g. another tubular or rod element mounted perpendicularly to the Arm 2240, although other angles of attachment in and out of the plane of the Arm 2240 may be employed within other embodiments of the invention.

Mounted to the Mount 2250 is a second Hub 2220 to which a Platform 2260 is attached. Accordingly, the second Hub 2220 allows the position along the Mount 2150 of the Platform 2260 to be varied as well as the rotational angle of the Platform 2260 relative to the Mount 2250. Similarly, first Hub 2210 allows for vertical positioning of the Platform 2260 up and down the Pole 2230. Optionally, additional Hubs may be employed to provide additional degrees of freedom such that with a combination of Hubs position of an element in respect of one, two, or three axes may be achieved in addition with rotation about these axes.

Within the embodiments of the invention described and depicted above POLSUS with one, two or three poles have been described and depicted. However, in some instances the pole configuration may not support mounting of all elements, whether it is one, two or three poles. This may, for example, be a lack of space for additional hangers for patient multiple fluid requirements or space to hold pumps, particularly the larger horizontal infusion pumps which are becoming more prevalent within many medical environments. Accordingly, the inventor has established a side arm for use in combination with a pole which becomes particularly significant in critical, intensive, acute and surgical care environments where patients require multiple pumps in addition to multiple fluids such as blood transfusion, chemotherapy, IV, dialysis, etc. The projections are also important in specific hospital laboratory requirements such as catheterization laboratories. With some POLSUS being required to hold additional medical equipment such as oxygen canisters and blood warmers the provisioning of side arm projections provides a valuable option for both accessories and their requisite liquids/pumps as well as the other elements described above.

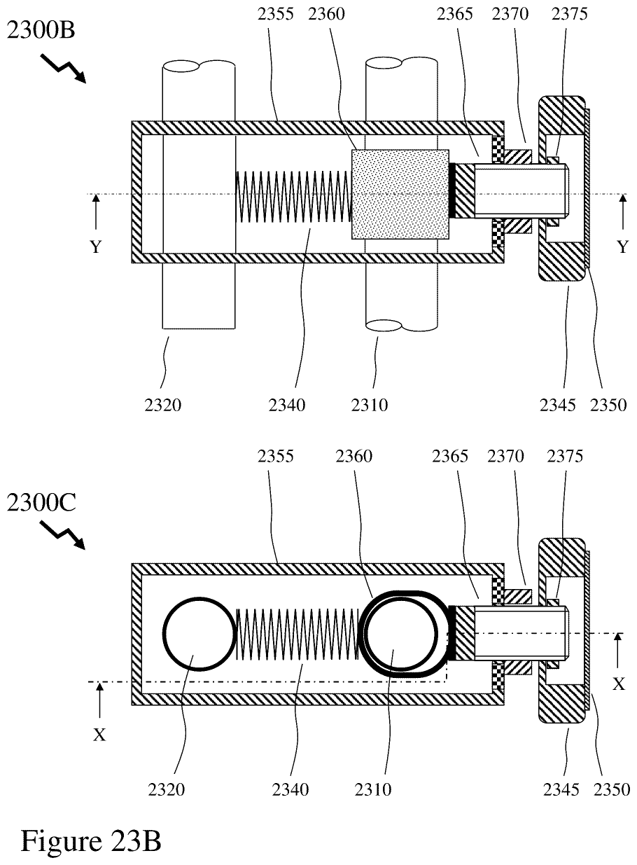

Accordingly, referring to FIGS. 23A and 23B there is depicted a side arm projection for use with POLSUS according to embodiments of the invention. Referring to FIG. 23A the first image 2300A depicts the Auxiliary Pole Mount 2330 with the Main Pole 2310, e.g. a pole connected to a base, and Auxiliary (Aux) Pole 2320. In common with the connection hubs described and depicted above in respect of FIGS. 21 and 22 the Auxiliary Pole Mount 2330 may employ an adjustable resistance knob as described and depicted in FIGS. 20A and 20B. First image 2300B in FIG. 23B depicts the Auxiliary Pole Mount 2330 in cross-sectional view along the section line X-X depicted in third image 2300C. Third image 2300C being along the section line Y-Y depicted in the second image 2300B.

Accordingly, referring to second image 2300B there is depicted the Main Pole 2310 to which the Aux Pole 2320 is mounted via the Auxiliary Pole Mount 2330. As depicted the Auxiliary Pole Mount 2330 comprises:
Body Portion 2355;
Knob 2345;
Cover 2350;
Threaded Mount 2365;
Nut 2370;
Locknut 2375;
Strap 2360; and
Spring 2340.

As depicted the Knob 2345 is attached to the Threaded Mount 2365 via Locknut 2375. The Nut 2370 is attached to the Body Portion 2355. Accordingly, rotation of the Knob 2345 rotates the Threaded Mount 2365 relative to the Nut 2370 such that the Threaded Mount 2365 moves left and right with reference to second Image 2300B or towards/away from the Main Pole 2310. Accordingly, this movement of the Threaded Mount 2365 also moves the Strap 2360. Considering anticlockwise movement of the Knob 2350 for conventional right hand threaded Threaded Mount 2365 and Nut 2370 then the Thread Mount 2365 moves away from the Main Pole 2310 wherein the Strap 2360 engages against the Main Pole 2310 and further rotation of the Knob 2345 engages the Strap 2360 further restraining the Auxiliary Pole Mount 2330 into position vertically on the Main Pole 2310. With the Strap 2360 and smooth exterior of the Main Pole 2310 rotation of the Auxiliary Pole Mount 2330 on the Main Pole 2310 is still possible. Within other embodiments of the invention the interior surface of the Strap 2360 and exterior surface of the Main Pole 2310 may prevent rotation of the Auxiliary Pole Mount 2330 once the Strap 2360 is tightened under action of the Knob 2345.

The Cover 2350 covers the opening within Knob 2345 to prevent ingress, improve cleanliness etc. and may be a snap fit, cold welded, or attached via an adhesive, resin etc. The Aux Pole 2320 is held in a vertical position under action of the Spring 2340. This allows a user to move the Aux Pole 2320 vertically relative to the Auxiliary Pole Mount 2330 which may be beneficial as in some instances the positioning of the Auxiliary Pole Mount 2330 may be restricted by other elements attached to the Main Pole 2310. Accordingly, if the Strap 2360 is loosened to allow the Auxiliary Pole Mount 2330 to be moved on the Main Pole 2310 the Aux Pole 2320 is retained relative to the Auxiliary Pole Mount 2330 under the action of the Spring 2340 and will move with the Auxiliary Pole Mount 2330.

The Strap 2360 may be formed from a sheet or tube of a rigid polymer. An inner surface of the Strap 2360 may be textured and/or coated to improve friction between inner surface of the Strap 2360 and the exterior surface of the Main Pole 2310.

Within other embodiments of the invention the Aux Pole 2320 may be rigidly mounted to the Auxiliary Pole Mount 2330 such that movement of it relative to the Auxiliary Pole Mount 2330 is not possible.

Operation of the Strap 2360 being evident in third image 2300C along the section line Y-Y within second image 2300B. Accordingly, as depicted a user can position the Auxiliary Pole Mount 2330 onto a pole, e.g. Mail Pole 2310 with a simple action wherein the Auxiliary Pole Mount 2330 and Aux Pole 2320 are retained in position by the action of the Spring 2340 upon the user releasing the Auxiliary Pole Mount 2330 and/or Aux Pole 2320 there were holding to move the Auxiliary Pole Mount 2330 upon the Main Pole 2310. The Strap 2360 and its associated tightening through the action of the Knob 2345 once the Auxiliary Pole Mount 2330 has been positioned provides additional retention of the Auxiliary Pole Mount 2330 to the Main Pole 2310.

However, it would be evident within embodiments of the invention that a sufficiently strong Spring 2340 means that the Strap 2360 etc. are not required at all. However, users derive psychological confidence by their use of the Knob 2345. Within other embodiments of the invention the Knob 2345 may be moved from a first position, e.g. engaged against the Auxiliary Pole Mount 2330 for example, to a second position, e.g. not engaged against the Auxiliary Pole Mount 2330 for example, and vice-versa but the Knob 2345 does not directly or indirectly engaged the Main Pole 2310 so that there Knob 2345 is a feature providing psychological benefit to the user without any physical impact on the retention of the Auxiliary Pole Mount 2330 to the Main Pole 2310 or retention of the Auxiliary Pole Mount 2330 to the Aux Pole 2320.

Optionally, a second Knob 2345 may be provided at the other end of the Auxiliary Pole Mount 2330 to provide a similar psychological impact for the positioning/retention of the Aux Pole 2320.

Optionally, a second assembly comprising another set of Knob 2345, Cover 2350, Threaded Mount 2365, Nut 2370, Locknut 2375 and Strap 2360 may be disposed to the other end of the Auxiliary Pole Mount 2330 to engage the Aux Pole 2320.

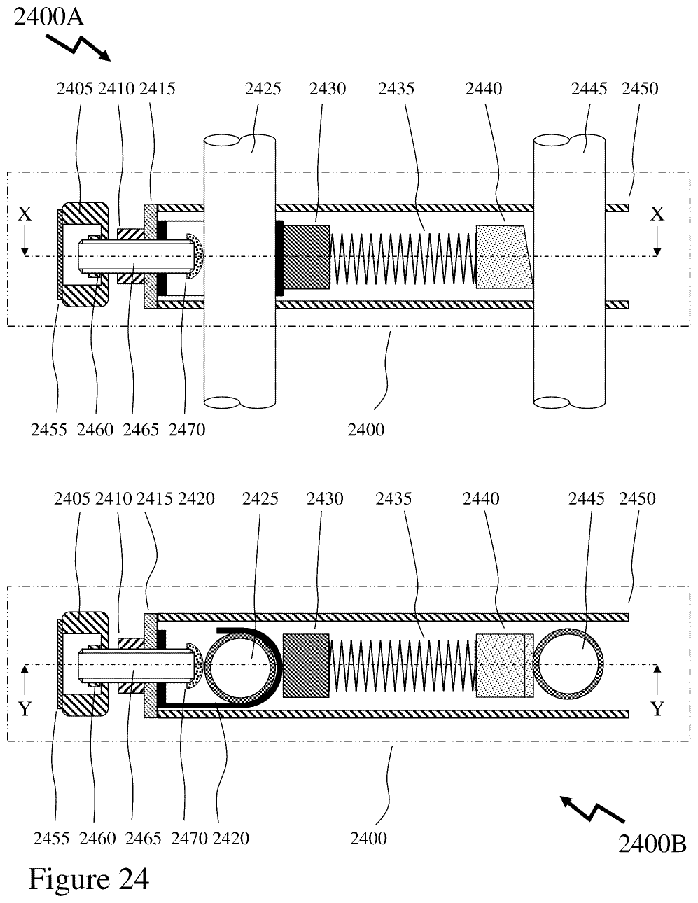

Now referring to FIG. 24 there is depicted another side arm to support an auxiliary pole for use with pole systems according to embodiments of the invention in a cross-sectional front elevation in first Image 2400A and a cross-sectional plan elevation in second Image 2400B which are along section lines Y-Y and X-X respectively. The Support 2400 being depicted as attaching to a Pole 2425 and supporting an Auxiliary (Aux) Pole 2445 and comprising:
Knob 2405
Nut 2410
Retainer Plate 2415
Retainer 2420
Block 2430
Spring 2435
Auxiliary (Aux) Block 2440
Body 2450
Cover 2455
Locknut 2460
Threaded Mount 2465; and
Engagement Plate 2470.

As depicted the Knob 2405 is attached to the Threaded Mount 2465 via Locknut 2460. The Nut 2410 is attached to the Body Portion 2450. Accordingly, rotation of the Knob 2405 rotates the Threaded Mount 2465 relative to the Nut 2410 such that the Threaded Mount 2465 moves left and right with reference to first Image 2400A or towards/away from the Pole 2425. Accordingly, this movement of the Threaded Mount 2465 moves the Engagement Plate 2470. Considering anticlockwise movement of the Knob 2405 for conventional right hand threaded Threaded Mount 2465 and Nut 2410 then the Thread Mount 2465 moves away from the Pole 2425 wherein the Engagement Plate 2470 disengages from the Pole 2425 allowing the Support 2400 to be moved vertically on the Pole 2425. A Retainer 2420 is attached to a Retainer Plate 2415 and surrounds part of the Pole 2425, this Retainer 2420 being formed from a rigid material. With clockwise movement of the Knob 2405 for a conventional right hand threaded Threaded Mount 2465 and Nut 2410 then the Thread Mount 2465 moves towards the Pole 2425 wherein the Engagement Plate 2470 engages the Pole 2425 pushing the Pole 2425 against the inner surface of the Retainer 2420 which is rigid and hence the Support 2400 is held in place vertically on the Pole 2425.

With a circular Pole 2425 the Support 2400 can be rotated in either locked or unlocked positions. Optionally, an engagement means on Engagement Plate 2470 and profile on Pole 2425 may restrict rotation of the Support 2400 on the Pole 2425 or limit the positioning to a number of predetermined positions around the Pole 2425.

Disposed sequentially away between the Pole 2425 and Aux Pole 2445 are Block 2430, Spring 2435, and Aux Block 2440. The Block 2430 engages against the outer surface of the Retainer 2420 whilst the Aux Block 2440 engages against the Aux Pole 2440 with the Spring 2435 maintaining pressure between the Block 2430/Pole 2425 and Aux Pole 2440/Aux Pole 2445 independent of the engagement between the Engagement Plate 2470 and the Pole 2430. Accordingly, once the Support 2400 is mounted onto the Pole 2425 and the Aux Pole 2445 attached, which may be eased through the profile depicted on the Aux Block 2440 allowing the Aux Pole 24445 to be inserted downwards pushing the Aux Block 2440 and compressing the Spring 2435. The Pole 2425 and Aux Pole 2455 are inserted into holes within the Body 2450. Hence, pressure generated by the Spring 2435 pushes the Block 2430 towards the Pole 2425 and the Aux Pole 2440 against the Aux Pole 2445. As the Aux Pole 2445 is through the Support 2400 the Aux Pole 2445 is pushed against the Portion 2450 and accordingly, the Aux Pole 2445 is retained with respect to the Support 2400 independent of the retention of the Support 2400 against the Pole 2425.

Referring back to FIG. 6 it was shown that through the design of the POLSUS base that multiple POLSUS could be nested together closely reducing storage space requirements etc. However, it would be evident that this close nesting may be problematic if equipment is still mounted to them such that either another pole impacts this equipment or other equipment on another pole impacts this equipment. Accordingly, the inventors have established what they refer to as an “ant-nester” to prevent POLSUS bases nesting together when undesired. Accordingly, referring to FIG. 25A there are depicted non-deployed and deployed configurations for an anti-nesting element for use with pole systems according to embodiments of the invention in first and second images 2500A and 2500B respectively. Referring initially to first Image 2500A then first to fourth POLSUS 2510A, 2510B, 2510C and 2510D are depicted nested as they were in FIG. 6. However, mounted to each is an Anti-Nester, namely first to fourth Anti-Nester 2520A, 2520B, 2520C and 2520D respectively, in a retracted position or non-deployed position wherein, considering first Anti-Nester 2520A it is parallel to the main cross-bar, e.g. Cross Bar 660 in FIG. 6, such that another POLSUS can be nested close if there is no issue with equipment etc. A reason for the nesting capability is through the design of the base which allows bases to enter a nesting configuration or be removed from a nested set of bases from either end or in either direction.

Now referring to second Image 2500B then the Anti-Nester 2520 is in a non-retracted position or deployed position wherein it is at an angle to the main cross-bar, e.g. Cross Bar 660 in FIG. 6, of the POLSUS 2530A such that another POLSUS 2530B cannot be nested close to it. However, as will become evident with respect to FIG. 26 the Anti-Nester 2520 may be configured to prevented nesting of the POLSUS but also linking them such that movement of one moves others coupled to it via the Anti-Nester.

Referring to FIG. 25B there are depicted first to fourth Elevations 2500C to 2500F of an exemplary design of an anti-nesting element according to an embodiment of the invention. These elevations being an end elevation, plan elevation, front elevation and bottom elevation. As depicted the Anti-Nester is formed from a channel with a Central Opening 2550 for the Anti-Nester to be mounted onto a pole, a first Engagement 2540A at its left end and a second Engagement 2540B at its right end which engage with other poles. The first Engagement 2540A and second Engagement 2540B being depicted as semi-circular recesses but it would be evident that other geometries may be employed without departing from the scope of the invention. Optionally, the first Engagement 2540A and second Engagement 2540B may be lateral bars across the width of the POLSUS, a clamp or spring clip to engage another pole etc. As depicted the bottom of the Anti-Nester has a slot formed within the two side pieces at an angle. The width of this slot being that of the main cross-bar of the POLSUS, e.g. Cross Bar 660, such that when in the deployed configuration the Anti-Nester sits down over the main cross-bar and engages with it limiting its rotation. This slot may be omitted within other embodiments of the invention. The width of the Anti-Nester from these two sides is similarly dimensioned such that the Anti-Nester in the non-deployed configuration and sit down over the cross-bar. Within embodiments of the invention the length of the Anti-Nester may be varied according to whether the POLSUS has a single isolated pole or multiple poles, for example.

FIGS. 26A and 26B depicts other exemplary designs of an anti-nesting element according to an embodiment of the invention. As depicted in FIG. 26A the Anti-Nester is depicted prior to forming as a sheet wherein post-stamping or alternate manufacturing processes the sides are folded, such folds being depicted by the virtual fold lines 2640A and 2640B such that when folded the Anti-Nester comprises a first Side 2620A, a Top 2620B and second Side 2620C. Disposed at each are first and second Pole Grips 2610A and 2610B for engaging with the pole(s) of the other POLSUS.

FIG. 26B depicts a similarly design Anti-Nester but now there are first and second Slots 2650A and 2650B within the second side 2620C and third and fourth Slots 2660A and 2660B within the first side 2620A. These slots, when the first side 2620A and second side 2620C are folded relative to the Top 2620B, engaging features upon the cross-bar of the POLSUS to retain the Anti-Nester within position at a predetermined angle relative to the cross-bar, e.g. 90°.

Within embodiments of the invention the poles have been described as being employed for the support of pole supports and other elements such as electrical distribution panels, handles etc. However, it would be evident that within other embodiments of the invention rather than being a single rod that a single tube may be employed. Optionally, the interior of a tubular pole (post) may provide additional functionality. For example, within an embodiment of the invention the pole may contain a series of batteries to provide short-term emergency DC power to equipment attached to the pole. The series of batteries may be connected to an electrical socket or electrical distribution panel on the POLSUS wherein in the event of a power failure the DC power from the battery stack is available. Within other embodiments of the invention this battery stack within a pole may employ rechargeable batteries which are trickle charged from an electrical socket or electrical distribution panel on the POLSUS.

Within other embodiments of the invention a tubular post may allow an electrical cable or cables to be run through the middle of the tube allowing power to be routed up a POLSUS. For example, a power cable may be routed from the bottom to a banner at the top of the POLSUS to provide power for a wireless interface, sensors, display etc. Optionally, multiple cables could be run exiting the POLSUS at different vertical positions in combination or independent of the top of the POLSUS. Each exit of a cable may provide protection/retention of the cable and terminate with a plug or socket at either end. Optionally, a power distribution board may be inserted into a POLSUS to provide a single power cable to connect to an electrical mains outlet and distribute to multiple outlets.

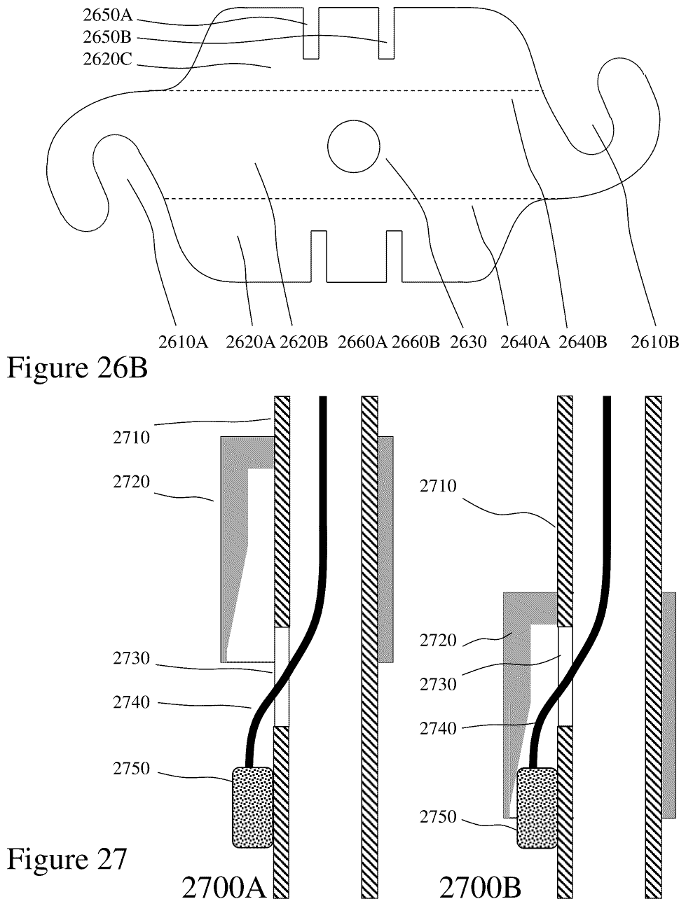

Referring to FIG. 27 there is depicted exemplary power entry/exit with cover for distributing power up a POLSUS according to an embodiment of the invention. In first Image 2700A the Cover 2720 is raised whilst an electrical Cable 2740 is inserted through an Opening 2730 with the Pole 2710. A Connector 2750 may be attached when the Cable 2740 is inserted or attached subsequent to the insertion of the Cable 2740 into the Opening 2730 and Pole 2710. In second Image 2700B the Cover 2720 has been lowered so that it covers the Opening 2730. The inner surface of the Cover 2720 being shaped to engage the Connector 2750 and push it against the Pole 2710 so that the movement of the Connector 2750 is restricted or eliminated. Optionally, the Cover 2750 may be dimensioned to simply push the Cable 2740 against the outer surface of the Pole 2710 as it is lowered.

Within other embodiments of the invention a pole (post) may be tubular allowing one or more electrical cables to be disposed within the pole. By replacing the solid bolts, for example bolts 1030 in FIGS. 10 and 11 with threaded and flanged tubular elements the one or more electrical cables can be fed through the base such that no cables are above the level of the base. Optionally, the one or more electrical cables may terminate within a socket formed within or attached to the pole at a predetermined position or may be coupled to an electrical distribution board (panel). Optionally, the one or more electrical cables may run the length of the pole to a cap or caps disposed upon the top of each pole, such as cap elements 820A and 820B in FIG. 8 or cap element 720 in FIG. 7. Such cap elements may connect to a display unit allowing messaging to be displayed at the top of a system employing these poles. This display unit may be wirelessly connected to provide for dynamic adjustment of the messaging displayed. Optionally, a cap element may provide emergency lighting. Optionally, a cap element may be coloured through one or more optical sources within the cap element such that systems associated with different departments, e.g. MRI, surgery, etc. may be distinguished by this colour. Optionally, the cap element may support multiple colours wherein a current colour is associated either with its currently assigned department and/or a status of a unit mounted to the system which is wirelessly connected to the cap element. In this manner, if a unit mounted to the system triggers an alarm in addition to its own alarms a visible indication of the system to which the alarming unit is associated is provided. This may be important in instances where a single patient is associated with two or more systems according to embodiments of the invention.

Whilst within the embodiments of the invention described above it is implied, assumed or stated that the post is round as the openings within the post supports are circular it would be evident to one of skill in the art that other opening geometries and post geometries may be employed without departing from the scope of the invention.

Within embodiments of the invention a shell or body portion of a post support may be formed from a metal, e.g. stainless steel, aluminum, etc.; an alloy, e.g. brass; a ceramic, e.g. alumina; or a plastic, e.g. nylon, polyphenylsulfone (PPSF or PPSU), polypropylene, polycarbonate, and polyoxymethylene. The shell or body portion may be uncoated or it may be coated with one or more materials including, but not limited to, an antibacterial agent, a resilient coloured powder coating, a paint, and an elastomer.

Within embodiments of the invention a fitting, e.g. a FLSH fitting or hook, may be formed from a metal, e.g. stainless steel, aluminum, etc.; an alloy, e.g. brass; a ceramic, e.g. alumina; or a plastic, e.g. nylon, polyphenylsulfone (PPSF or PPSU), polypropylene, polycarbonate, and polyoxymethylene. The fitting may be uncoated or it may be coated with one or more materials including, but not limited to, an antibacterial agent, a resilient coloured powder coating, a paint, and an elastomer.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

1. The method according to claim 28, further comprising:
a non-removable knob assembly comprising:
a fixing which terminates with a threaded portion at one end;
a knob comprising a body with a first opening through the knob such that the knob fits over the threaded portion of the fixing, a second opening disposed at a first end of the knob nearest the terminal end of the threaded portion of the fixing, and a threaded portion disposed at a second distal end to the first end;
a locknut attached to the terminal end of the threaded portion; wherein
rotation of the knob in a first direction results in the knob moving along the threaded portion of the fixing away from the terminal end of the threaded portion of the fixing;
rotation of the knob in a second direction results in the knob moving along the threaded portion of the fixing towards the terminal end of the threaded portion of the fixing; and
the locknut limits movement of the knob when rotated in the second direction.

2. The method according to claim 1, wherein
the non-removable knob assembly has either a first configuration or a second configuration;
in the first configuration the non-removable knob assembly further comprises:
a flange at a second distal end of the fixing from the end with the threaded portion; wherein
the flange retains the fixing against a first element; and
rotation of the knob in the first direction into contact with a second element opposite the first element applies pressure to the second element via the knob and the first element via the flange of the fixing;
in the second configuration the non-removable knob assembly further comprises:
a flange at a second distal end of the fixing from the end with the threaded portion; wherein
the flange retains the fixing against a first element;
rotation of the knob in the first direction into contact with a second element opposite the first element applies pressure to the second element via the knob and the first element via the flange of the fixing;
a first surface of the first element distal to a second surface of the first element which engages with the flange of the fixing is profiled to fit around at least one of a tube with a predetermined external geometry and a rod with the predetermined external geometry; and
a second surface of the second element distal to a second surface of the second element which engages with the knob is profiled to fit around at least one of a tube with a predetermined external geometry and a rod with the predetermined external geometry.

3. (canceled)

4. The method according to claim 28, further comprising
a mount comprising:
a body with a threaded opening through which a threaded element passes;
a knob attached to a first end of the threaded element;
a strap disposed within the body attached to a second distal end of the threaded element and surrounding a first pole passing through the mount;
the strap is tightened to retain the mount in position through rotation of the knob moving the threaded element relative to the body;
the strap is loosened through rotation of the knob moving the threaded element relative to the body thereby allowing the mount to be moved upon the first pole.

5. The method according to claim 4, further comprising at least one of:
a spring disposed between the pole and another pole which passes through the mount wherein the spring retains the another pole in position relative to the mount independent of movement of threaded element and tightening or loosening of the strap around the pole; and
a further pole attached to the mount.

6. The method according to claim 4, wherein
either:
an inner surface of the strap and an outer surface of the pole allow the mount to be rotated around the pole even when the strap is tightened to retain the mount in position vertically upon the pole;
or:
an inner surface of the strap and an outer surface of the pole are profiled to prevent the mount being rotated around the pole even when the strap is tightened to retain the mount in position vertically upon the pole.

7-8. (canceled)

9. The method according to claim 28, further comprising:
a body with a threaded opening through which a threaded element passes, a pair of openings for the pole to fit through the body and another pair of openings for another pole to fit through the body;
a knob attached to a first end of the threaded element;
an engagement plate attached at a second distal end of the threaded element;
a retainer attached to the body at one end and having a predetermined geometry at a second distal end; and
a spring disposed between a block and an auxiliary block; wherein
operation of the knob moves the engagement plate relative to the retainer to either hold or release a first pole inserted through the pair of openings and disposed between the engagement plate and an inner surface of the retainer; and
the block, spring and auxiliary block retain the another pole inserted through the other pair of openings independent of the position of the knob.

10. (canceled)

11. The method according to claim 28, further comprising:
a pole support comprising:
a cross-bar;
a first bar with a pair of wheels disposed towards opposite ends of the first bar;
a second bar with another pair of wheels disposed towards opposite ends of the second bar; and
an anti-nester element comprising a body with a hole for mounting the anti-nester element to the pole; wherein,
the cross-bar is mounted to the pole;
in a first configuration with the anti-nester element parallel to the cross-bar the pole support can be nested with another pole support;
in a second configuration with the anti-nester element at a predetermined angle to the cross-bar the pole support cannot be nested with another pole support.

12. The method according to claim 11, further comprising
a first fitting at one end of the anti-nester element to engage another pole of another pole support; and
a second fitting at another distal end of the anti-nester element to engage a further pole of the further pole support.

13. The method according to claim 11, wherein
either:
the anti-nester element further comprises an opening in one or more lower surfaces;
in the first configuration the anti-nester element fits over the cross-bar; and
in the second configuration the opening fits over the cross-bar to fix the anti-nester element at the predetermined angle;
or:
the anti-nester element further comprises an opening in one or more lower surfaces;
in the first configuration the anti-nester element fits over the cross-bar; and
in the second configuration the opening fits over a feature on an upper surface of the cross-bar to fix the anti-nester element at the predetermined angle.

14. (canceled)

15. The method according to claim 28, further comprising:
providing a support;
providing a shell having an opening of predetermined diameter disposed at a first end to fit over the pole;
providing a fitting disposed at a second distal end to provide a specific function; and
providing a split ring disposed within a groove around the opening within the shell; wherein
the split ring projects into the opening and has an inner opening having a diameter established in dependence upon the diameter of the pole; and
the support is fitted onto the pole by inserting the pole through the opening and is retained in position on the pole independent of any other means or element.

16. The method according to claim 15, wherein
at least one of:
either the groove is formed by inserting an insert into the opening and retaining the insert in position within the shell by at least one of an interference fit, welding or a material attaching the insert to the shell or the groove is formed through injection molding the shell with the split-ring in position; and
in a first position with the second distal end of the support is lower than the first end of the support the support is retained in position independent of any other means or element and in a second position with the second distal end of the support is level with the first end of the support the support the support can be moved on the pole.

17. The method according to claim 15, further comprising
providing a projection upon a portion of the groove such that the projection projects into a gap portion of the split ring such that the split ring cannot rotate within the groove.

18. (canceled)

19. The method according to claim 28, further comprising:
providing a support comprising:
a shell having an opening disposed at a first end to fit over a pole;
a fitting disposed at a second distal end to provide a specific function; and
an O-ring disposed within the opening within the shell; wherein
the opening has a dimension along an axis of the shell from the first end to the second end established in dependence upon a diameter of the pole and a thickness of the shell;
the O-ring projects into the opening;
the support can be moved relative to the pole by the user lifting the second distal end of the support to a position level with the first end of the support; and
the support is retained in position when the user releases the second distal end of the support such that is lower than the first end of the support.

20. The method according to claim 19, wherein
at least one of:
the support is retained in position on the pole independent of any other means or element; and
the O-ring is retained in position within the shell by an insert and the insert is retained in position by at least one of a material and via an interference fit.

21. (canceled)

22. The base according to claim 28, further comprising:
a first arm for attaching to a bar in a first predetermined location;
a second arm for attaching to the bar in a second predetermined location;
the bar having one or more mounting points;
a first pair of wheels attached to third predetermined locations relative to each end of the first arm;
a second pair of wheels attached to fourth predetermined locations relative to each end of the second arm; and
a third pair of wheels attached to fifth predetermined locations relative to each end of the arm; wherein
the third predetermined locations, the fourth predetermined locations, and fifth predetermined locations are all points on a common circle; and
each of the one or more mounting points is for mounting a pole.

23. The base according to claim 22, wherein
the base is one of a plurality of bases; and
the plurality of bases can be nested together; wherein
for each base of the plurality of bases other than a last base of the plurality of bases in a nested set of bases (nest) the first ends of each of the first arm and second arm fit under the bar of a next base of the plurality of bases in the nest;
for each base of the plurality of bases other than a first base in the plurality of bases in the nest the second distal ends of each of the first arm and second arm fit under the bar of a preceding base of the plurality of bases in the nest; and
the bar of each base of the plurality of bases other than a last base of the plurality of bases is in contact with the bar of the preceding base of the plurality of bases;
the bar of each base of the plurality of bases other than a first base of the plurality of bases is in contact with the bar of the next base of the plurality of bases.

24. The base according to claim 22, further comprising
a buffer mounted to at least one of the base and one or more first poles attached to the one or more mounting points; wherein
the buffer limits the distance between the base and another base such that first items mounted to the one or more first poles of the base do not hit or are not hit by second items mounted to one or more second poles attached to the one or more mounting points on the other base.

25. (canceled)

26. The method according to claim 28, wherein
providing the base comprises:
providing a first arm for attaching to a bar in a first predetermined location;
providing a second arm for attaching to the bar in a second predetermined location;
providing the bar having one or more mounting points;
providing a first pair of wheels attached to third predetermined locations relative to each end of the first arm;
providing a second pair of wheels attached to fourth predetermined locations relative to each end of the second arm; and
providing a third pair of wheels attached to fifth predetermined locations relative to each end of the arm; wherein
the third predetermined locations, the fourth predetermined locations, and fifth predetermined locations are all points on a common circle; and
each of the one or more mounting points is for mounting a pole.

27. The method according to claim 28, wherein
providing the support comprises:
providing a shell having an opening disposed at a first end to fit over a pole;
providing a fitting disposed at a second distal end to provide a specific function; and
providing an O-ring disposed within the opening within the shell; wherein
the opening has a dimension along an axis of the shell from the first end to the second end established in dependence upon a diameter of the pole and a thickness of the shell;
the O-ring projects into the opening;
the support can be moved relative to the pole by the user lifting the second distal end of the support to a position level with the first end of the support; and
the support is retained in position when the user releases the second distal end of the support such that is lower than the first end of the support.

28. A method relating to a pole support system comprising:
providing a base; and
providing a pole attached to the base.
